SEQUENCE LISTING (5 sequences)

SEQ ID NO: 1 - protein, 309 aa, Homo sapiens
MNGTYNTCGSSDLTWPPAIKLGFYAYLGVLLVLGLLLNSLALWVFCCRMQQWTETRIYMTNLAVADLCLLCTLPFVLHSLRDTSDTPLCQLSQGIYLTNRYMSISLVTAIAVDRYVAVRHPLRARGLRSPRQAAAVCAVLWVLVIGSLVARWLLGIQEGGFCFRSTRHNFNSMAFPLLGFYLPLAVVVFCSLKVVTALAQRPPTDVGQAEATRKAARMVWANLLVFVVCFLPLHVGLTVRLAVGWNACALLETIRRALYITSKLSDANCCLDAICYYYMAKEFQEASALAVAPSAKAHKSQDSLCVTLA

SEQ ID NO: 2 - DNA, 1875 bp, Homo sapiens
caggccagagtcccagctgtcctggactctgctgtggggaagggctgatgcaggtgtggagtcaaatgtgggtgcctcctgcagccgggtgccaggaggggtggaggggccaccctgggctttgtccgggagcctggtcttcccgtccttgggctgacaggtgctgctgcctctgagccctccctgctaagagctgtgtgctgggtaaggctggtggccctttgggctccctgtccaggatttgtgctctggagggtagggcttgctgggctggggactggaggggaacgtggagctccttctgcctcctttcctgccccatgacagcaggcagatcccaggagagaagagctcaggagatgggaagaggatctgtccaggggttagacctcaagggtgacttggagttctttacggcacccatgctttctttgaggagttttgtgtttgtgggtgtggggtcggggctcacctcctcccacatccctgcccagaggtgggcagagtgggggcagtgccttgctccccctgctcgctctctgctgacctccggctccctgtgctgccccaggaccatgaatggcacctacaacacctgtggctccagcgacctcacctggcccccagcgatcaagctgggcttctacgcctacttgggcgtcctgctggtgctaggcctgctgctcaacagcctggcgctctgggtgttctgctgccgcatgcagcagtggacggagacccgcatctacatgaccaacctggcggtggccgacctctgcctgctgtgcaccttgcccttcgtgctgcactccctgcgagacacctcagacacgccgctgtgccagctctcccagggcatctacctgaccaacaggtacatgagcatcagcctggtcacggccatcgccgtggaccgctatgtggccgtgcggcacccgctgcgtgcccgcgggctgcggtcccccaggcaggctgcggccgtgtgcgcggtcctctgggtgctggtcatcggctccctggtggctcgctggctcctggggattcaggagggcggcttctgcttcaggagcacccggcacaatttcaactccatggcgttcccgctgctgggattctacctgcccctggccgtggtggtcttctgctccctgaaggtggtgactgccctggcccagaggccacccaccgacgtggggcaggcagaggccacccgcaaggctgcccgcatggtctgggccaacctcctggtgttcgtggtctgcttcctgcccctgcacgtggggctgacagtgcgcctcgcagtgggctggaacgcctgtgccctcctggagacgatccgtcgcgccctgtacataaccagcaagctctcagatgccaactgctgcctggacgccatctgctactactacatggccaaggagttccaggaggcgtctgcactggccgtggctcccagtgctaaggcccacaaaagccaggactctctgtgcgtgaccctcgcctaagaggcgtgctgtgggcgctgtgggccaggtctcgggggctccgggaggtgctgcctgccaggggaagctggaaccagtagcaaggagcccgggatcagccctgaactcactgtgtattctcttggagccttgggtgggcagggacggcccaggtacctgctctcttgggaagagagagggacagggacaagggcaagaggactgaggccagagcaaggccaatgtcagagacccccgggatggggcctcacacttgccacccccagaaccagctcacctggccagagtgggttcctgctggccagggtgcagccttgatgacacctgccgctgcccctcggggctggaataaaactccccacccagagtc

SEQ ID NO: 3 - DNA, 930 bp, Homo sapiens
atgaatggcacctacaacacctgtggctccagcgacctcacctggcccccagcgatcaagctgggcttctacgcctacttgggcgtcctgctggtgctaggcctgctgctcaacagcctggcgctctgggtgttctgctgccgcatgcagcagtggacggagacccgcatctacatgaccaacctggcggtggccgacctctgcctgctgtgcaccttgcccttcgtgctgcactccctgcgagacacctcagacacgccgctgtgccagctctcccagggcatctacctgaccaacaggtacatgagcatcagcctggtcacggccatcgccgtggaccgctatgtggccgtgcggcacccgctgcgtgcccgcgggctgcggtcccccaggcaggctgcggccgtgtgcgcggtcctctgggtgctggtcatcggctccctggtggctcgctggctcctggggattcaggagggcggcttctgcttcaggagcacccggcacaatttcaactccatggcgttcccgctgctgggattctacctgcccctggccgtggtggtcttctgctccctgaaggtggtgactgccctggcccagaggccacccaccgacgtggggcaggcagaggccacccgcaaggctgcccgcatggtctgggccaacctcctggtgttcgtggtctgcttcctgcccctgcacgtggggctgacagtgcgcctcgcagtgggctggaacgcctgtgccctcctggagacgatccgtcgcgccctgtacataaccagcaagctctcagatgccaactgctgcctggacgccatctgctactactacatggccaaggagttccaggaggcgtctgcactggccgtggctcccagtgctaaggcccacaaaagccaggactctctgtgcgtgaccctcgcctaa

SEQ ID NO: 4 - protein, 340 aa, Homo sapiens [sequence omitted]

SEQ ID NO: 5 - DNA, 1023 bp, Homo sapiens
atgctgagtggttcccgggctgtccccactccacaccgtggcagtgaagagctgctgaagtacatgcttcatagtccttgcgtctctctgaccatgaatggcacctacaacacctgtggctccagcgacctcacctggcccccagcgatcaagctgggcttctacgcctacttgggcgtcctgctggtgctaggcctgctgctcaacagcctggcgctctgggtgttctgctgccgcatgcagcagtggacggagacccgcatctacatgaccaacctggcggtggccgacctctgcctgctgtgcaccttgcccttcgtgctgcactccctgcgagacacctcagacacgccgctgtgccagctctcccagggcatctacctgaccaacaggtacatgagcatcagcctggtcacggccatcgccgtggaccgctatgtggccgtgcggcacccgctgcgtgcccgcgggctgcggtcccccaggcaggctgcggccgtgtgcgcggtcctctgggtgctggtcatcggctccctggtggctcgctggctcctggggattcaggagggcggcttctgcttcaggagcacccggcacaatttcaactccatggcgttcccgctgctgggattctacctgcccctggccgtggtggtcttctgctccctgaaggtggtgactgccctggcccagaggccacccaccgacgtggggcaggcagaggccacccgcaaggctgcccgcatggtctgggccaacctcctggtgttcgtggtctgcttcctgcccctgcacgtggggctgacagtgcgcctcgcagtgggctggaacgcctgtgccctcctggagacgatccgtcgcgccctgtacataaccagcaagctctcagatgccaactgctgcctggacgccatctgctactactacatggccaaggagttccaggaggcgtctgcactggccgtggctcccagtgctaaggcccacaaaagccaggactctctgtgcgtgaccctcgcctaa

(19) United States
(12) Patent Application Publication
(10) Pub. No.: US 2013/0316985 A1
(43) Pub. Date: Nov. 28, 2013

(54) GPR35 Ligands And Uses Thereof

(71) Applicant: CORNING INCORPORATED, Corning, NY (US)

(72) Inventors: Huayun Deng, Painted Post, NY (US); Ye Fang, Painted Port, NY (US)

(73) Assignee: Corning Incorporated, Corning, NY (US)

(21) Appl. No.: 13/897,886

(22) Filed: May 20, 2013

Related U.S. Application Data
(60) Provisional application No. 61/651,693, filed on May 25, 2012.

(51) Int. Cl. A61K 31/655; A61K 31/192; A61K 31/235; A61K 31/381; A61K 31/24; A61K 31/175; A61K 31/06; A61K 31/05; A61K 31/055

(52) U.S. Cl. CPC A61K 31/655; A61K 31/05; A61K 31/192; A61K 31/235; A61K 31/055; A61K 31/24; A61K 31/175; A61K 31/06; A61K 31/381
USPC 514/150; 514/734; 568/763; 514/568; 562/475; 514/544; 560/61; 514/735; 568/765; 514/542; 560/29; 514/590; 564/20; 514/583; 564/36; 514/728; 564/441; 564/328; 564/315; 534/660; 549/50; 549/57; 514/443

(57) ABSTRACT

A pharmaceutical composition including at least one compound of the Formulas (I), (II), or (III), or a pharmaceutically acceptable salt, solvate, clathrate, or prodrug thereof, as defined herein. Also disclosed is a method of treatment of diseases which are pathophysiologically related to GPR35, the GPR35-hERG signaling complex, or both, as defined herein.

### CROSS-REFERENCE TO RELATED APPLICATIONS

This application claims the benefit of priority under 35 U.S.C. §119 of U.S. Provisional Application Ser. No. 61/651,693, filed on May 25, 2012, the content of which is relied upon and incorporated herein by reference in its entirety.

This application is related to commonly owned and assigned patent application number U.S. Ser. No. 13/182,019, to Fang, Y., et al., entitled “Compositions and methods for the treatment of pathological conditions related to GPR35 and/or GPR35-hERG complex”; and U.S. Ser. No. 61/515,409, to Deng, H., et al., entitled “GPR35 ligands and the uses thereof,” but does not claim priority thereto.

The entire disclosure of any publication or patent document mentioned herein is incorporated by reference.

### SEQUENCE LISTING

This application contains a Sequence Listing electronically submitted via EFS-Web to the United States Patent and Trademark Office as text filed named “SP12-119_SequqnceListing_ST25.txt” having a size of 11 kb and created on Mar. 25, 2013. Due to the electronic filing of the Sequence Listing, the electronically submitted Sequence Listing serves as both the paper copy required by 37 CFR §1.821(c) and the CRF required by §1.821(e). The information contained in the Sequence Listing is hereby incorporated herein by reference.

### BACKGROUND

G protein-coupled receptor-35 (GPR35) is an orphan G protein-coupled receptor (GPCR), and has been implicated to play a role in heart failure and hypertension, coronary artery disease, asthma, pain, early-onset inflammatory bowel disease, inflammation (see MacKenzie, A. E., et al., GPR35 as a novel therapeutic target, Front. Endocrin. 2011, 2, e68). GPR35 is expressed in a variety of tissues including human immune cells, pancreas, small intestine, colon, and spleen. Up-regulation of GPR35 has been found in human failing heart, and several types of inflamed immune cells. These findings have led to speculation that GPR35 represents a novel potential therapeutic target.

Several GPR35 agonists have been reported in literature, which agonists include several clinically used drugs. Cromolyn and dicumarol, the two anti-asthma drugs with unknown mechanism of action, were recently identified to be GPR35 agonists with moderate potency (see Yang, Y., et al., G-protein-coupled receptor 35 is a target of the asthma drugs cromolyn disodium and nedocromil sodium, Pharmacology, 2010, 86, 1-5). Bumetanide and furosemide, two loop diuretics used for treating cardiovascular disease, were also found to be GPR35 agonists (see Yang, Y., et al., GPR35 is a target of the loop diuretic drugs bumetanide and furosemide, Pharmacology, 2012, 89, 13-17). GPR35 was also found to be a target of entacapone, a catechol-O-methyl transferase inhibitor drug for the treatment of Parkinson's disease (see Deng, H., et al., Tyrphostin analogs are GPR35 agonists, FEBS Lett., 2011, 585, 1957-1962), and the anti-nociception niflumic acid and certain abundant natural phytochemicals including myricetin, morin, and ellagic acid (see Deng, H., et al., Discovery of natural phenols as G protein-coupled receptor-35 (GPR35) agonists, ACS Med. Chem. Lett., 2012, 3, 165-169), and gallic acid and certain aspirin metabolites (see Deng, H., et al., (2012) Anti-inflammatory gallic acid and wedelolactone are G protein-coupled receptor-35 agonists, Pharmacology, 89:211-219; Deng, H., et al., Aspirin metabolites are GPR35 agonists, Naunyn-Schmiedeberg's Archives of Pharmacology, 2012, DOI 10.1007/s00210-012-07). These findings provide rationales for the concept of the activation of GPR35 as a therapeutic target in certain diseases including inflammatory disorders (see Deng, H., et al., Thieno[3,2-b]thiophene-2-carboxylic Acid Derivatives as GPR35 Agonists, Bioorg. Med. Chem. Lett., 2012, DOI 10.1016/j.bmcl.2012.04.057).

New drug therapies for the treatment of subjects suffering from or susceptible to pathological conditions or diseases associated with GPR35 would be highly beneficial.

### SUMMARY

In embodiments, the disclosure provides a method for treatment or prevention of a disease, comprising:
administering, to a subject diagnosed as in need of such treatment or prevention, an effective amount of a compound of the Formula (I), (II), or (III):

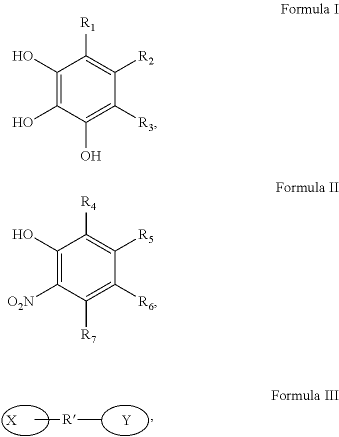

where:
R¹, R², R³, R⁴, R⁵, R⁶, and R⁷ are each individually selected from —H, —NO₂, —C(═O)OH, —C(═O)OR″ where R″ is substituted or unsubstituted alkyl having from 1 to 20 carbon atoms, halide, acyl halide, substituted or unsubstituted alkyl, substituted or unsubstituted —NH-alkyl, substituted or unsubstituted —CH₂—NH—C(═O)—R″, substituted or unsubstituted —CH═NH—NH—C(═O)—NH—R″, substituted or unsubstituted —CH═NH—NH—C(═S)—NH—R″, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, or a substituted or unsubstituted alkoxy;

optionally R² and R³ taken together form a substituted or unsubstituted aryl, substituted or unsubstituted heteroaryl, substituted or unsubstituted cycloalkenyl, or substituted or unsubstituted heterocyclyl;

optionally R⁴ and R⁵, R⁵ and R⁶, or R⁶ and R⁷, taken together form a substituted or unsubstituted aryl, substituted or unsubstituted heteroaryl, substituted or unsubstituted cycloalkenyl or substituted or unsubstituted heterocyclyl;

in Formula (III), R′ is a divalent moiety selected from: a covalent carbon-carbon bond, —N═N-(cis- or trans-), —NH—NH—, —O—, —(CH₂CH₂O)ₙ— or —(CH₂CH(—CH₃)—O)ₙ— where n is from 1 to 10, substituted or unsubstituted alkyl, substituted or unsubstituted —NH-alkyl, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, substituted or unsubstituted alkoxy, or of the formulas —CH═CH—C(═O)—CH═C(—OH)—CH═CH— (conjugated keto-enol form) or —CH═CH—C(═O)—O—CH₂—CH₂— (conjugated ester);

in Formula (III), X and Y are independently selected from substituents of the Formulas (IV), (V), (VI), (VII), (VIII), or (IX):

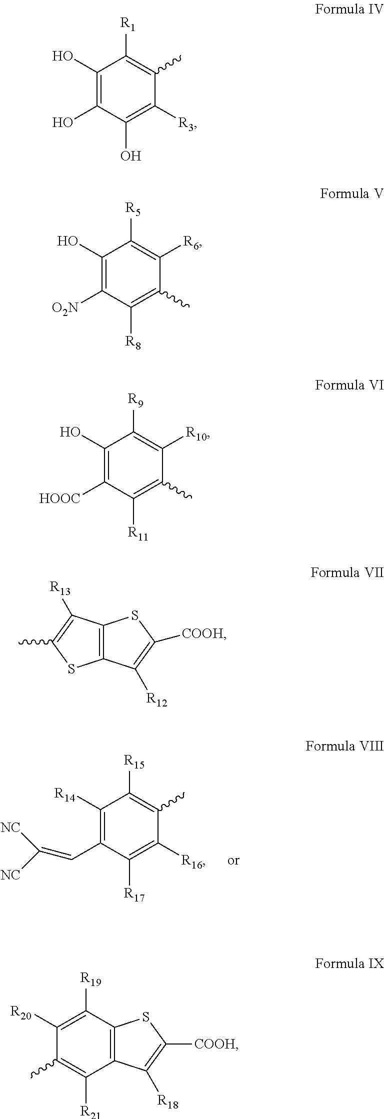

where
the wavy or squiggly line represents a connecting valence to R′ or to another of X or Y when R′ is a single covalent carbon-carbon bond, and in any of the Formulas (IV), (V), (VI), (VII), (VIII), or (IX), each R¹, R³, R⁵, R⁶, R⁸, R⁹, R¹⁰, R¹¹, R¹², R¹³, R¹⁴, R¹⁵, R¹⁶, R¹⁷, R¹⁸, R¹⁹, R²⁰, and R²¹ is independently selected from —H, —OH, halide, acyl halide, substituted or unsubstituted alkyl, substituted or unsubstituted —NH-alkyl, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, or a substituted or unsubstituted alkoxy; or a pharmaceutically acceptable salt, solvate, clathrate, or prodrug thereof.

In embodiments, the disclosure provides a pharmaceutical composition including a compound of the abovementioned Formulas (I), (II), or (III), or a pharmaceutically acceptable salt, solvate, clathrate, or prodrug thereof, as defined herein.

In compounds of Formula (I) or Formula (I), in embodiments, R1, R2, R3, R4, R5, R6, and R7 can independently be, for example, selected from —H, —NO2, —C(═O)OH, —C(═O)OR″ where R″ is substituted or unsubstituted alkyl having from 1 to 20 carbon atoms, halide, acyl halide, substituted or unsubstituted alkyl, substituted or unsubstituted —NH-alkyl, substituted or unsubstituted —CH2-NH—C(═O)—R″, substituted or unsubstituted —CH═NH—NH—C(═O)—NH—R″, substituted or unsubstituted —CH═NH—NH—C(═S)—NH—R″, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, or a substituted or unsubstituted alkoxy;

In Formula (III), in embodiments, X and Y can be the same or different. X and Y can independently be, for example, of the Formulas (IV), (V), (VI), (VII), (VIII), or (IX):

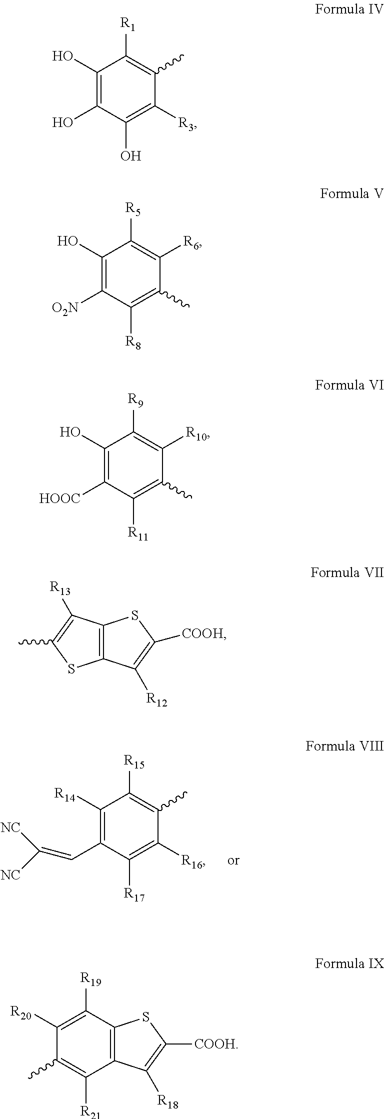

where the wavy or squiggly line represents a connecting valence to R′ or to another of X or Y when R′ is a single covalent carbon-carbon bond.

In embodiments, in Formula (III), R′ is a divalent moiety selected from, for example, a single covalent carbon-carbon bond joining X and Y, —N═N-(cis or trans), —NH—NH—, —O—, —(CH2CH2O)n-, or —(CH2CH(—CH3)-O)n- where n is from 1 to 10, substituted or unsubstituted alkyl, substituted or unsubstituted —NH-alkyl, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, substituted or unsubstituted alkoxy, or of the formulas —CH═CH—C(═O)—CH═C(—OH)—CH═CH— (conjugated keto-enol form) or —CH═CH—C(═O)—O—CH2-CH2- (conjugated ester).

In embodiments, the disclosure provides a method for treatment or prevention of a disease, comprising: administering, to a subject diagnosed as in need of such treatment or prevention, an effective amount of a compound of the aforementioned Formula (I), (II), or (III), or a pharmaceutically acceptable salt, solvate, clathrate, or prodrug thereof, where the substituents are as defined herein.

In embodiments, the disclosure provides a method of treatment for reducing the risk of certain diseases, treating certain diseases, or both, which diseases are pathophysiologically related to GPR35.

In embodiments, the disclosed treatment or prevention methods can administer to a human or animal subject one or more compounds of a class of compounds of the abovementioned Formulas (I), (II), or (III), including a pharmaceutically acceptable salt, solvate, clathrate, or prodrug thereof, where the substituents are as defined herein.

In embodiments, the disclosure provides a compound selected from the abovementioned Formula (I), (II), (III), and mixtures thereof, or a pharmaceutically acceptable salt, solvate, clathrate, or prodrug thereof. These compounds are useful, alone or in combination, as therapeutic agents for modulating GPR35, and for therapeutic prevention or treatment of diseases to which GPR35 activity is pathophysiologically related.

The disclosed compounds can be GPR35 modulators. The compounds can be useful as therapeutic agents for modulating GPR35, and for treatment or reducing the risk of diseases that are related to the activity of GPR35.

Additional features and advantages are set forth in the detailed description.

The foregoing general description and the detailed description are merely exemplary, and are intended to provide an overview or framework to understanding the nature and character of the claims. The accompanying drawings, if any, are included to provide a further understanding. The drawings illustrate one or more embodiment(s), and together with the description serve to explain principles and operation of the various embodiments.

### BRIEF DESCRIPTION OF THE DRAWINGS

FIGS. 1A to 1F demonstrate that a series of di-nitrophenol analog compounds act as a GPR35 agonist.

FIGS. 2A to 2D show the dose-dependent DMR responses of a series of dinitrophenol analogs in HT29 cells.

FIGS. 3A to 3D show that the known GPR35 antagonist ML145 dose-dependently inhibited the DMR signals induced by nitrophenol compounds in HT29.

FIGS. 4A to 4D show the dose-dependent beta-arrestin translocation responses for a series of dinitrophenol analogs in U2OS-GPR35-bla cell line as measured using the Tango beta-arrestin assay.

FIG. 5 shows that the known GPR35 antagonist ML145 dose-dependently inhibited the Tango beta-arrestin signals induced by selected nitrophenol compounds in U2OS-GPR35-bla cell line.

### DETAILED DESCRIPTION

The disclosed compounds, compositions, articles, devices, and methods are not limited to specific synthetic methods or specific treatment methods unless otherwise specified, or to particular reagents unless otherwise specified. The terminology used herein is for the purpose of describing particular embodiments only and is not intended to be limiting.

In embodiments, the disclosure provides pharmaceutical compositions including an effective amount of a compound of any of the aforementioned Formulas (I), (II), (III), or a pharmaceutically acceptable salt, solvate, clathrate, or prodrug thereof; and a pharmaceutically acceptable carrier or vehicle. These compositions can further include additional agents. These compositions are useful for modulating the activity of GPR35, and can improve the prevention and the treatment of GPR35 associated human diseases, such as metabolic disorders.

The disclosed methods also encompass methods for treating or reducing the risk of GPR35 associated human diseases such as metabolic disorders and cancers, including administering to a subject a compound of the Formulas (I), (II), (III), or a pharmaceutically acceptable salt, solvate, clathrate, or prodrug thereof. The disclosed methods can also include administering to the subject an additional agent separately or in a combination composition with the compound of the Formulas (I), (II), (III), or a pharmaceutically acceptable salt, solvate, clathrate, or prodrug thereof.

Also disclosed are methods for treating GPR35 associated human diseases such as metabolic disorders and cancers, such as be administering in vivo or in vitro a compound of the Formula (I), (II), (III), or a pharmaceutically acceptable salt, solvate, clathrate, or prodrug thereof. A compound of the Formula (I), (II), (III), or a pharmaceutically acceptable salt, solvate, clathrate, or prodrug thereof can be administered to a subject in a therapeutically effective amount.

### A. G PROTEIN-COUPLED RECEPTORS (GPCRs)

G protein coupled receptors are intrinsic membrane proteins which comprise a large superfamily of receptors. It has been estimated that one percent of human genes may encode GPCRs. Many GPCRs share a common molecular architecture and common signaling mechanism. Historically, GPCRs have been classified into six families, originally thought to be unrelated, three of which are found in vertebrates. Recent work has identified several new GCPR families and suggested the possibility of a common evolutionary origin for all of them.

One characteristic feature of most GPCRs is that seven clusters of hydrophobic amino acid residues, or transmembrane regions (TMs, the 7 transmembrane (7TM) regions are designated as TM1, TM2, TM3, TM4, TMS, TM6, and TM7) are located in the primary structure and pass through (span) the cell membrane at each region thereof. The domains are believed to represent transmembrane alpha-helices connected by three intracellular loops (i1, i2, and i3), three extracellular loops (e1, e2, and e3), and amino (N)- and carboxyl (C)-terminal domains (Palczewski et al., Science 289, 739-45 (2000)). Most GPCRs have single conserved cysteine residues in each of the first two extracellular loops which form disulfide bonds that are believed to stabilize functional protein structure. It is well known that these structures detailed above are common among G protein coupled receptor proteins and that the amino acid sequences corresponding to the area where the protein passes through the membrane (membrane-spanning region or transmembrane region) and the amino acid sequences near the membrane-spanning region are often highly conserved among the receptors. Thus, due to the high degree of homology in GPCRs, the identification of novel GPCRs, as well identification of both the intracellular and the extracellular portions of such novel members, is readily accomplished by those of skill in the art.

1. GPR35

GPR35 is a rhodopsin-like GPCR first identified in 1998 (O'Dowd, et al., Genomics 47: 310-313 (1998)). GPR35 was first identified to an orphan GPCR that contains 309 amino acids. GPR35b, a splicing variant that contains an N-terminal extension of 31 amino acids, was later discovered in gastric cancer cells in 2004, and shown to be capable of transforming NIH-3T3 cells (Okumura, et al., Cancer Sci. 95: 131-135 (2004)). GPR35 has been found to be expressed in various tissues including stomach, gastrointestinal tissues, and mast cells, basophils and eosinophils. Upregulation of GPR35 has also been identified in human mast cells upon challenge with IgE antibodies, in human macrophages after exposure to benzo(α)pyrene, in failing heart cells, and in gastric cancer cells.

Identification of ligands, particularly the endogenous ligands, that activate GPR35 are desired. To date, there are several agonists for GPR35 reported so far, including kynurenic acid, NPPB, zaprinast, pamoic acid and lysophosphatidic acid (LPA). Both kynurenic acid and LPA are indicated to be an endogenous ligand for GPR35 (Wang, et al., J. Biol. Chem. 281: 22021-22028 (2006); Oka, et al., Biochem. Biophys. Res. Comm. 395: 232-237 (2010)). Both kynurenic acid and LPA elicited several cellular responses in HEK293 cells and/or CHO cells expressing GPR35. For example, in HEK-293 cells expressing GPR35, 2-acyl LPA markedly enhanced the Ca2+ response, the activation of RhoA and the phosphorylation of ERK in GPR35-expressing cells. 2-Acyl LPA also induced the internalization of the receptor molecules. Nevertheless, it remains unclear whether kynurenic acid or LPA is the natural agonist for GPR35. Recently using a GPR35-β-arrestin-2 interaction assay Jenkins et al., discovered a number of compounds possessing agonist activity on GPR35. These agonists include cromolyn disodium, dicumarol, pamoate, niflumic acid, and luteolin. These compounds active at human GPR35 in the β-arrestin-2 interaction assay were also able to promote cell growth via Gα13 (Jenkins, et al., Biochemical Journal 432, 451-459 (2010)).

New drug are sought and are highly desirable in therapies for the treatment of subjects suffering from or susceptible to pathological conditions or diseases associated with GPR35. In particular, new drugs having one or more improved properties, such as safety profile, efficacy, or physical properties, relative to those drugs currently available would be highly desirable.

### B. DEFINITIONS

Throughout this application, various publications are referenced. The disclosures of these publications in their entireties are hereby incorporated by reference into this application to more fully describe the state of the art to which this pertains. The references disclosed are also individually and specifically incorporated by reference herein for the material contained in them that is discussed in the sentence in which the reference is relied upon.

1. A, an, the

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

2. Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

3. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

4. Analytical Methods

An analytical method is for example, a method which measures a molecule or substance. For example, gas chromatography, gel permeation chromatography, high resolution gas chromatography, high resolution mass spectrometry, or mass spectrometry is analytical methods.

5. Assaying

Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

6. Assaying the Response

“Assaying the response” or like terms means using a means to characterize the response. For example, if a molecule is brought into contact with a cell, a biosensor can be used to assay the response of the cell upon exposure to the molecule.

7. Agonism Action

Agonism action refers to the binding of a molecule to a receptor that leads to the activation of the receptor, thus triggering a cellular response similar to the cellular response for a known agonist for the receptor.

8. Antagonism Action

Antagonism action refers to the binding of a molecule to a receptor that leads to the inhibition of the receptor.

9. Agonism and Antagonism Mode

The agonism mode or like terms is the assay wherein the cells are exposed to a molecule to determine the ability of the molecule to trigger biosensor signals such as DMR signals, while the antagonism mode is the assay wherein the cells are exposed to a maker in the presence of a molecule to determine the ability of the molecule to modulate the biosensor signal of cells responding to the marker.

10. Anti-Inflammation Agent

An anti-inflammatory agent is any agent that has an anti-inflammatory activity. Examples of anti-inflammation agent are Cox inhibitors such as ibuprofen, aspirin, Tylenol, or GPR35 agonists, or GPR35-hERG complex activators.

11. Anti-Metabolic-Disorder Agent

An anti-metabolic disorder agent is any agent that has an effect in suppressing, reducing, or preventing diseases associated with metabolic disorders. Metabolism is the process the human body uses to get or make energy from the food. Food is made up of proteins, carbohydrates and fats. Chemicals in digestive system break the food parts down into sugars and acids, thus providing fuels. The body can use this fuel right away, or it can store the energy in tissues, such as liver, muscles and body fat. A metabolic disorder occurs when abnormal chemical reactions in human body disrupt this process. When this happens, one might have too much of some substances or too little of other ones that one needs to stay healthy.

12. Anti-Congestive-Heart-Failure Agent

An anti-congestive heart failure agent is any agent that has an effect in suppressing, reducing, or preventing diseases associated with congestive heart failure, such as a diuretic, a particular compound known in the art is ramipril, which is an angiotensin-converting enzyme (ACE) inhibitor that is used to treat high blood pressure and congestive heart failure.

13. Anti-Cancer Agent

An anti-cancer agent is any agent that has an anti-cancer effect, such as vinblastine or taxol.

14. Biosensor

Biosensor or like terms refer to a device for the detection of an analyte that combines a biological component with a physicochemical detector component. The biosensor typically consists of three parts: a biological component or element (such as tissue, microorganism, pathogen, cells, or combinations thereof), a detector element (works in a physicochemical way such as optical, piezoelectric, electrochemical, thermometric, or magnetic), and a transducer associated with both components. The biological component or element can be, for example, a living cell, a pathogen, or combinations thereof. In embodiments, an optical biosensor can comprise an optical transducer for converting a molecular recognition or molecular stimulation event in a living cell, a pathogen, or combinations thereof into a quantifiable signal. Typical biosensors used for label-free cellular assays are surface plasmon resonance, plasmon resonance imaging, resonant waveguide grating biosensor, photonic crystal biosensor, and electric impedance biosensors.

15. Biosensor Response

A “biosensor response”, “biosensor output signal”, “biosensor signal” or like terms is any reaction of a sensor system having a cell to a cellular response. A biosensor converts a cellular response to a quantifiable sensor response. A biosensor response is an optical response upon stimulation as measured by an optical biosensor such as surface plasmon resonance (SPR) or resonant waveguide grating (RWG) biosensor or it is a bioimpedence response of the cells upon stimulation as measured by an electric biosensor. Since a biosensor response is directly associated with the cellular response upon stimulation, the biosensor response and the cellular response can be used interchangeably, in embodiments of the disclosure.

16. Biosensor Signal

A “biosensor signal” or like terms refers to the signal of cells measured with a biosensor that is produced by the response of a cell upon stimulation.

17. Cell

The term “cell” as used herein also refers to individual cells, cell lines, or cultures derived from such cells. A “culture” refers to a composition comprising isolated cells of the same or a different type. The term co-culture is used to designate when more than one type of cell are cultured together in the same dish with either full or partial contact with each other. A cell can be a recombinantly engineered cell wherein the cell comprises exogenous nucleic acid.

Cell refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

18. Cell Culture

“Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

19. Cell Panel

A “cell panel” or like terms is a panel which comprises at least two types of cells. The cells can be of any type or combination disclosed herein.

20. Cellular Response

A “cellular response” or like terms is any reaction by the cell to a stimulation.

21. Cellular Process

A cellular process or like terms is a process that takes place in or by a cell. Examples of cellular process include, but not limited to, proliferation, apoptosis, necrosis, differentiation, cell signal transduction, polarity change, migration, or transformation.

22. Cellular Target

A “cellular target” or like terms is a biopolymer such as a protein or nucleic acid whose activity can be modified by an external stimulus. Cellular targets are most commonly proteins such as enzymes, kinases, ion channels, and receptors.

23. Characterizing

Characterizing or like terms refers to gathering information about any property of a substance, such as a ligand, molecule, marker, or cell, such as obtaining a profile for the ligand, molecule, marker, or cell.

24. Chemistry Definitions

The term “alkyl” refers to a linear or branched saturated hydrocarbyl substituent (i.e., a substituent obtained from a hydrocarbon by removal of a hydrogen) containing from one to twenty carbon atoms; in an embodiment from one to twelve carbon atoms; in another embodiment, from one to ten carbon atoms; in another embodiment, from one to six carbon atoms; and in another embodiment, from one to three carbon atoms. Examples of such substituents include methyl, ethyl, propyl (including n-propyl and isopropyl), butyl (including n-butyl, isobutyl, sec-butyl and tert-butyl), pentyl, iso-amyl, hexyl, and like substituents.

The term “alkenyl” refers to a linear or branched hydrocarbyl substituent containing one or more double bonds and from two to twenty carbon atoms; in another embodiment, from two to twelve carbon atoms; in another embodiment, from two to six carbon atoms; and in another embodiment, from two to four carbon atoms. Examples of alkenyl include ethenyl (also known as vinyl), allyl, propenyl (including 1-propenyl and 2-propenyl) and butenyl (including 1-butenyl, 2-butenyl and 3-butenyl). The term “alkenyl” includes substituents having “cis” and “trans” orientations, or alternatively, “E” and “Z” orientations.

The term “alkynyl” refers to a linear or branched hydrocarbyl substituent containing one or more triple bonds and from two to twenty carbon atoms; in another embodiment, from two to twelve carbon atoms; in another embodiment, from two to six carbon atoms; and in another embodiment, from two to four carbon atoms. Examples of alkynyl include ethynyl, propynyl, butyryl, pentynyl, hexynyl, methylpropynyl, 4-methyl-1-butynyl, 4-propyl-2-pentynyl-, and 4-butyl-2-hexynyl.

The term “benzyl” refers to methyl radical substituted with phenyl, i.e., —CH2-C6H5.

The term “carbocyclic ring” refers to a saturated cyclic, partially saturated cyclic, or aromatic ring containing from 3 to 14 carbon ring atoms (“ring atoms” are the atoms bound together to form the ring). A carbocyclic ring typically contains from 3 to 10 carbon ring atoms. Examples include cyclopropyl, cyclobutyl, cyclopentyl, cyclopentenyl, cyclopentadienyl, cyclohexyl, cyclohexenyl, cyclohexadienyl, and phenyl. A “carbocyclic ring system” alternatively may be 2 or 3 rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluorenyl, and decalinyl.

The term “heterocyclic ring” refers to a saturated cyclic, partially saturated cyclic, or aromatic ring containing from 3 to 14 ring atoms (“ring atoms” are the atoms bound together to form the ring), in which at least one of the ring atoms is a heteroatom that is oxygen, nitrogen, or sulfur, with the remaining ring atoms being independently selected from the group consisting of carbon, oxygen, nitrogen, and sulfur. A heterocycloalkyl alternatively may comprise 2 or 3 rings fused together, wherein at least one such ring contains a heteroatom as a ring atom (e.g., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heterocyclic ring” also includes substituents that are fused to a C6-C10 aromatic ring or to a 5-10-membered heteroaryl, wherein a group having such a fused heterocyclic group as a substituent is bound to a heteroatom of the heterocyclic group or to a carbon atom of the heterocycloalkyl group. When such a fused heterocycloalkyl group is substituted with one more substituents, the one or more substituents, unless otherwise specified, are each bound to a hetero atom of the heterocyclocalkyl group or to a carbon atom of the heterocyclic group. The fused C6-C10 aryl ring or to a 5-10-membered heteroaryl ring may be optionally substituted with halogen, C1-C6 alkyl, C3-C10 cycloalkyl, or ═O

The term “cycloalkyl” refers to a saturated carbocyclic substituent having three to fourteen carbon atoms. In embodiments, a cycloalkyl substituent has three to ten carbon atoms. Examples of cycloalkyl include cyclopropyl, cyclobutyl, cyclopentyl and cyclohexyl. The term “cycloalkyl” also includes substituents that are fused to a C6-C10 aromatic ring or to a 5-10-membered heteroaromatic ring, wherein a group having such a fused cycloalkyl group as a substituent is bound to a carbon atom of the cycloalkyl group. When such a fused cycloalkyl group is substituted with one or more substituents, the one or more substituents, unless otherwise specified, are each bound to a carbon atom of the cycloalkyl group. The fused C6-C10 aromatic ring or to a 5-10-membered heteroaromatic ring may be optionally substituted with halogen, C1-C6 alkyl, C3-C10 cycloalkyl, or ═O.

The term “cycloalkenyl” refers to a partially unsaturated carbocyclic substituent having three to fourteen carbon atoms, typically three to ten carbon atoms. Examples of cycloalkenyl include cyclobutenyl, cyclopentenyl, and cyclohexenyl.

A cycloalkyl or cycloalkenyl may be a single ring, which typically contains from 3 to 6 ring atoms. Examples include cyclopropyl, cyclobutyl, cyclopentyl, cyclopentenyl, cyclopentadienyl, cyclohexyl, cyclohexenyl, cyclohexadienyl, and phenyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C4-C10 carbocyclic ring, such as a C5 or a C6 carbocyclic ring, or to a 4-10-membered heterocyclic ring, wherein a group having such a fused aryl group as a substituent is bound to an aromatic carbon of the aryl group. When such a fused aryl group is substituted with one more substituents, the one or more substituents, unless otherwise specified, are each bound to an aromatic carbon of the fused aryl group. The fused C4-C10 carbocyclic or 4-10-membered heterocyclic ring may be optionally substituted with halogen, C1-C6 alkyl, C3-C10 cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring.

The term “hydrogen” refers to hydrogen substituent, and may be depicted as —H.

The term “hydroxy” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “hydroxyalkyl” refers to an alkyl that is substituted with at least one hydroxy substituent. Examples of hydroxyalkyl include hydroxymethyl, hydroxyethyl, hydroxypropyl and hydroxybutyl.

The term “nitro” means —NO2.

The term “cyano” (also referred to as “nitrile”)—CN, which also may be depicted: —C≡N.

The term “carbonyl” means —C(═O)—.

The term “amino” refers to —NR2. An amino group can be a primary, secondary or tertiary amino group. Each R in —NR2 can individually be —H, alkyl, alkenyl, alkynykl, aryl, heteroaryl, cycloalkyl or heterocyclyl.

The term “alkylamino” refers to an amino group, wherein at least one alkyl chain is bonded to the amino nitrogen in place of a hydrogen atom. Examples of alkylamino substituents include monoalkylamino such as methylamino (exemplified by the formula —NH(CH3)), and dialkylamino such as dimethylamino, (exemplified by the formula —N(CH3)2).

The term “aminocarbonyl” means —C(═O)—NH2.

The term “halogen” refers to fluorine (—F), chlorine (—Cl), bromine (—Br), or iodine (—I). In embodiments, the halogen is chlorine or fluorine.

The prefix “halo” indicates that the substituent to which the prefix is attached is substituted with one or more independently selected halogen substituents. For example, haloalkyl refers to an alkyl that is substituted with at least one halogen substituent. Where more than one hydrogen is replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 2,2,2-trifluoroethyl, difluoroethyl, pentafluoroethyl, difluoropropyl, dichloropropyl, and heptafluoropropyl. Illustrating further, “haloalkoxy” refers to an alkoxy that is substituted with at least one halogen substituent. Examples of haloalkoxy substituents include chloromethoxy, 1-bromoethoxy, fluoromethoxy, difluoromethoxy, trifluoromethoxy (also known as “perfluoromethyloxy”), and 2,2,2-trifluoroethoxy. It should be recognized that if a substituent is substituted by more than one halogen substituent, those halogen substituents may be identical or different (unless otherwise stated).

The prefix “perhalo” indicates that each hydrogen substituent on the substituent to which the prefix is attached is replaced with an independently selected halogen substituent. If all the halogen substituents are identical, the prefix may identify the halogen substituent. Thus, for example, the term “perfluoro” means that every hydrogen substituent on the substituent to which the prefix is attached is replaced with a fluorine substituent. To illustrate, the term “perfluoroalkyl” refers to an alkyl substituent wherein a fluorine substituent is in the place of each hydrogen substituent. Examples of perfluoroalkyl substituents include trifluoromethyl (—CF3), perfluorobutyl, perfluoroisopropyl, perfluorododecyl, and perfluorodecyl. To illustrate further, the term “perfluoroalkoxy” refers to an alkoxy substituent wherein each hydrogen substituent is replaced with a fluorine substituent. Examples of perfluoroalkoxy substituents include trifluoromethoxy (—O—CF3), perfluorobutoxy, perfluoroisopropoxy, perfluorododecoxy, and perfluorodecoxy.

The term “oxo” refers to ═O.

The term “oxy” refers to an ether substituent, and may be depicted as —O—.

The term “alkoxy” refers to an alkyl linked to an oxygen, which may also be represented as —O—R, wherein the R represents the alkyl group. Examples of alkoxy include methoxy, ethoxy, propoxy and butoxy.

The term “alkylthio” means —S-alkyl. For example, “methylthio” is —S—CH3. Other examples of alkylthio include ethylthio, propylthio, butylthio, and hexylthio.

The term “alkylcarbonyl” means —C(═O)-alkyl. For example, “ethylcarbonyl” may be depicted as: —C(═O)—CH2-CH3. Examples of other alkylcarbonyl include methylcarbonyl, propylcarbonyl, butylcarbonyl, pentylcabonyl, and hexylcarbonyl.

The term “aminoalkylcarbonyl” means —C(═O)-alkyl-NH2. For example, “aminomethylcarbonyl” may be depicted as: —C(═O)—CH2-NH2.

The term “alkoxycarbonyl” means —C(═O)—O-alkyl. For example, “ethoxycarbonyl” may be depicted as: —C(═O)—O—CH2-CH3. Examples of other alkoxycarbonyl include methoxycarbonyl, ethoxycarbonyl, propoxycarbonyl, butoxycarbonyl, pentoxycarbonyl, and hexyloxycarbonyl. In embodiments, where the carbon atom of the carbonyl is attached to a carbon atom of a second alkyl, the resulting functional group is an ester.

The terms “thio” and “thia” mean a divalent sulfur atom and such a substituent may be depicted as —S—. For example, a thioether is represented as “alkyl-thio-alkyl” or, alternatively, alkyl-5-alkyl.

The term “thiol” refers to a sulfhydryl substituent, and may be depicted as —SH.

The term “thione” refers to ═S.

The term “sulfonyl” refers to —S(═O)2-. Thus, for example, “alkyl-sulfonyl-alkyl” refers to alkyl-S(═O)2-alkyl. Examples of alkylsulfonyl include methylsulfonyl, ethylsulfonyl, and propylsulfonyl.

The term “aminosulfonyl” means —S(═O)2-NH2.

The term “sulfinyl” or “sulfoxido” means —S(═O)—.

Thus, for example, “alkylsulfinylalkyl” or “alkylsulfoxidoalkyl” refers to alkyl-S(═O)-alkyl. Exemplary alkylsulfinyl groups include methylsulfinyl, ethylsulfinyl, butylsulfinyl, and hexylsulfinyl.

Examples of single-ring heteroaryls include furanyl, dihydrofuranyl, tetradydrofuranyl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiaodiazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

Examples of 2-fused-ring heteroaryls include, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include 5,6-dihydro-4H-imidazo[4,5,1-ij]quinoline, 4,5-dihydroimidazo[4,5,1-hi]indole, 4,5,6,7-tetrahydroimidazo[4,5,1-jk][1]benzazepine, and dibenzofuranyl.

Other examples of fused-ring heteroaryls include benzo-fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “heteroaryl” also includes substituents such as pyridyl and quinolinyl that are fused to a C4-C10 carbocyclic ring, such as a C5 or a C6 carbocyclic ring, or to a 4-10-membered heterocyclic ring, wherein a group having such a fused aryl group as a substituent is bound to an aromatic carbon of the heteroaryl group or to a heteroatom of the heteroaryl group. When such a fused heteroaryl group is substituted with one more substituents, the one or more substituents, unless otherwise specified, are each bound to an aromatic carbon of the heteroaryl group or to a heteroatom of the heteroaryl group. The fused C4-C10 carbocyclic or 4-10-membered heterocyclic ring may be optionally substituted with halogen, C1-C6 alkyl, C3-C10 cycloalkyl, or ═O.

A substituent is “substitutable” if it comprises at least one carbon, sulfur, oxygen or nitrogen atom that is bonded to one or more hydrogen atoms. Thus, for example, hydrogen, halogen, and cyano do not fall within this definition. If a substituent is described as being “substituted,” a non-hydrogen substituent is in the place of a hydrogen substituent on a carbon, oxygen, sulfur or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R,″ wherein R′ and R″ together with the nitrogen atom to which they are attached, may form a heterocyclic ring. The heterocyclic ring formed from R′ and R″ together with the nitrogen atom to which they are attached may be partially or fully saturated. In embodiments, the heterocyclic ring consists of 3 to 7 atoms. In embodiments, the heterocyclic ring is selected from the group consisting of pyrrolyl, imidazolyl, pyrazolyl, triazolyl, tetrazolyl, isoxazolyl, pyridyl and thiazolyl.

This specification uses the terms “substituent,” “radical,” and “group” interchangeably. If a group of substituents are collectively described as being optionally substituted by one or more of a list of substituents, the group may include: 1) unsubstitutable substituents; 2) substitutable substituents that are not substituted by the optional substituents; 3) substitutable substituents that are substituted by one or more of the optional substituents; or combinations thereof. If a substituent is described as being optionally substituted with up to a particular number of non-hydrogen substituents, that substituent may be either 1) not substituted; or 2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

When a substituent is comprised of multiple moieties, unless otherwise indicated, it is the intention for the final moiety to serve as the point of attachment to the remainder of the molecule. For example, in a substituent A-B-C, moiety C is attached to the remainder of the molecule. In a substituent A-B-C-D, moiety D is attached to the remainder of the molecule. Similarly, in a substituent aminocarbonylmethyl, the methyl moiety is attached to the remainder of the molecule, where the substituent may also be depicted as —CH2-C(═O)—NH2. In a substituent trifluoromethylaminocarbonyl, the carbonyl moiety is attached to the remainder of the molecule, where the substituent may also be depicted as —C(═O)—NH—CF3.

If substituents are described as being “independently selected” from a group, each substituent is selected independent of the other. Each substituent therefore may be identical to or different from the other substituent(s).

25. Compound Interchangeability

For the purposes of the present disclosure the terms “compound” and “composition of matter” stand equally well for the chemical entities described herein, including all enantiomeric forms, diastereomeric forms, salts, and the like, and the terms “compound” and “composition of matter” are used interchangeably throughout the present specification.

26. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed and a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

27. Contacting

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e., such that they are in proximity to mix or touch. For example, having a solution of composition A and cultured cell B and pouring solution of composition A over cultured cell B would be bringing solution of composition A in contact with cell culture B. Contacting a cell with a ligand would be bringing a ligand to the cell to ensure the cells have access to the ligand. Anything disclosed herein can be brought into contact with anything else. For example, a cell can be brought into contact with a marker or a molecule, a biosensor, and so forth.

28. Compounds and Compositions

Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if particular biological material, such as EGF is disclosed, EGF in its compound form is also disclosed.

29. Control

The terms “control” or “control levels” or “control cells” are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard.

30. Clathrate

A compound for use in the disclosure may form a complex such as a “clathrate”, a drug-host inclusion complex, wherein, in contrast to solvates, the drug and host are present in stoichiometric or non-stoichiometric amounts. A compound used herein can also contain two or more organic and/or inorganic components which can be in stoichiometric or non-stoichiometric amounts. The resulting complexes can be ionised, partially ionised, or non-ionised. For a review of such complexes, see J. Pharm. ScL, 64 (8), 1269-1288, by Haleblian (August 1975).

31. Congestive Heart Failure

Congestive heart failure (CHF) is a condition in which the heart's function as a pump to deliver oxygen rich blood to the body is inadequate to meet the body's needs. Congestive heart failure can be caused by diseases that weaken the heart muscle, or diseases that cause stiffening of the heart muscles, or diseases that increase oxygen demand by the body tissue beyond the capability of the heart to deliver. Many diseases can impair the pumping action of the ventricles. For example, the muscles of the ventricles can be weakened by heart attacks or infections (myocarditis). The diminished pumping ability of the ventricles due to muscle weakening is called systolic dysfunction. After each ventricular contraction (systole) the ventricle muscles need to relax to allow blood from the atria to fill the ventricles. This relaxation of the ventricles is called diastole. Diseases such as hemochromatosis or amyloidosis can cause stiffening of the heart muscle and impair the ventricles' capacity to relax and fill; this is referred to as diastolic dysfunction. The most common cause of this is longstanding high blood pressure resulting in a thickened (hypertrophied) heart. Additionally, in some patients, although the pumping action and filling capacity of the heart may be normal, abnormally high oxygen demand by the body's tissues (for example, with hyperthyroidism) may make it difficult for the heart to supply an adequate blood flow (called high output heart failure). In some patients one or more of these factors can be present to cause congestive heart failure. Congestive heart failure can affect many organs of the body. For example, the weakened heart muscles may not be able to supply enough blood to the kidneys, which then begin to lose their normal ability to excrete salt (sodium) and water. This diminished kidney function can cause to body to retain more fluid. The lungs may become congested with fluid (pulmonary edema) and the person's ability to exercise is decreased. Fluid may likewise accumulate in the liver, thereby impairing its ability to rid the body of toxins and produce essential proteins. The intestines may become less efficient in absorbing nutrients and medicines. Over time, untreated, worsening congestive heart failure will affect virtually every organ in the body.

32. Cancer

Cancer is a disease of inadequately controlled differentiation or division of cells, such as prostate cancer, leukemia, hormone dependent cancers, breast cancer, colon cancer, lung cancer, epidermal cancer, liver cancer, esophageal cancer, stomach cancer, cancer of the brain, and cancer of the kidney. Cancer is a collection of diseases that arise from the progressive accumulation of genetic alterations in somatic cells. Cancer is also viewed as a pathway dysregulated disease—a small number of core pathways are dominate in aberrant cell growth leading to cancer. The ability of tumor cells to outgrow their neighboring cells is often driven by constitutive activation of downstream proteins. Genetic studies over several decades have discovered a wide range of tumor-associated genes and their mutations, many of which preferentially occur in signaling proteins involved in a small number of pathways. Genetic mutations are often enriched in positive regulatory loops (gain of function), and methylated genes in negative regulatory loops (loss of function), leading to the disruption of the normal cooperative behavior of cells and thus promoting tumor phenotypes. A hallmark in the onset of cancer is how mutated proteins alter and govern signaling of cancer cells in the context of intracellular or intercellular signaling networks.

33. Detect

Detect or like terms refer to an ability of the apparatus and methods of the disclosure to discover or sense a molecule- or a marker-induced cellular response and to distinguish the sensed responses for distinct molecules.

34. Direct Action (of a Drug Candidate Molecule)

A “direct action” or like terms is a result (of a drug candidate molecule) acting independently on a cell.

35. DMR Signal

A “DMR signal” or like terms refers to the signal of cells measured with an optical biosensor that is produced by the response of a cell upon stimulation, see commonly owned and assigned U.S. Pat. No. 8,076,090.

36. DMR Response

A “DMR response” or like terms is a biosensor response using an optical biosensor. The DMR refers to dynamic mass redistribution or dynamic cellular matter redistribution. A P-DMR is a positive DMR response, a N-DMR is a negative DMR response, and a RP-DMR is a recovery P-DMR response.

37. Disease Marker

A disease marker is any reagent, molecule, substance, etc, that can be used for identifying, diagnosing, or prognosing for a GPR35 related disease.

38. Drug Candidate Molecule

A drug candidate molecule or like terms is a test molecule which is being tested for its ability to function as a drug or a pharmacophore. This molecule may be considered as a lead molecule.

39. Efficacy

Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue, or system level.

40. Electrophysiology Method

An electrophysiology method is any method which studies the electrical properties of biological cells and tissues. It involves measurements of voltage change or electric current on a wide variety of scales from single ion channel proteins to whole organs like the heart. In neuroscience, it includes measurements of the electrical activity of neurons, and particularly action potential activity. Recordings of large-scale electric signals from the nervous system such as electroencephalography, may also be referred to as electrophysiological recordings.

41. Engineered Cell

An engineered cell is any cell in which one or more genes have been added or removed (via genetic blockage, such as homologous recombination or siRNAa plasmid) or altered in either a transient or permanent fashion. The term “engineered cell” refers to a cell which has been manipulated to comprise exogenous material, such as nucleic acid. For example, disclosed herein are engineered cells which have been manipulated to comprise exogenous GPR35, exogenous hERG, or both.

42. Fusion Protein

A “fusion protein” is a protein or a peptide located either on the C- or N-terminal of the target protein, which facilitates one or several of the following characteristics: (1) improved solubility—Fusion of the N-terminus of the target protein to the C-terminus of a soluble fusion partner often improves the solubility of the target protein; (2) improved detection—Fusion of the target protein to either terminus of a short peptide (epitope tag) or protein which is recognized by an antibody (Western blot analysis) or by biophysical methods (e.g., GFP by fluorescence) facilitates the detection of the resulting protein during expression or purification; (3) improved purification—Simple purification schemes have been developed for proteins used at either terminus which bind specifically to affinity resins; (4) Localization—Tag, usually located on N-terminus of the target protein, which acts as address for sending protein to a specific cellular compartment; (5) improved Expression (E)—Fusion of the N-terminus of the target protein to the C-terminus of a highly expressed fusion partner results in high level expression of the target protein.

A “GPR35 fusion protein” refers to a protein or peptide located either on the C- or N-terminal of the target protein GPR35. Examples are GFP-GPR35 fusion protein that the green fluorescent protein (GFP) is located on the N-terminal of GPR35, while GPR35-GFP fusion protein that GFP is located on the C-terminal of GPR35. The tagged protein or peptide can also be located within the intracellular loops of the receptor.

43. GPR35 Modulator

A GPR35-specific modulator or GPR35 modulator or the like term is any modulator which directly binds to GPR35 and thus modulates the activity of GPR35. A typical GPR35-specific modulator can modulate GPR35 activity in one of three cellular assays: (1) Ca²⁺ mobilization assays in an engineered cell such as HEK-GPR35 with and without co-expressing G_{qo5}. G_{qo5} is a G protein whose activation results in Ca²⁺ mobilization, and the G_{qo5} protein can be activated by the agonist-induced activation of a non-G_{q}-coupled receptor when expressed in the cell. Since GPR35 is believed to be a non-G_{q}-coupled receptor, the co-expression of G_{qo5} is necessary to detect the GPR35 agonist induced Ca²⁺ mobilization signal. (2) Receptor internalization assays. Receptor internalization is quick and universal to almost all GPCRs. (3) Label-free dynamic mass redistribution (DMR) assays, as promised by optical biosensors such as resonant waveguide grating biosensor. The GPR35 modulator can be an agonist, an antagonist, an inverse agonist, or a biased agonism. Alternative assays such as beta-arrestin translocation assays or gene reporter assays can also be used.

44. GPR35 Expressing Cell

A GPR35 expressing cell is any cell which produces a functional GPR35 in the cell membrane of the cell.

45. GPR35 Agonist

A GPR35 agonist is any molecule which binds to and thus activates the GPR35 receptor in the cells. Examples, as disclosed herein or in published literature, include, but not limited to, diflunisal, flufenamic acid, flunxin, furosemide, niflumic acid, NPPB, tolfenamic acid, zaprinast, YE210, or DNQX.

46. GPR35 Antagonist

A GPR35 antagonist is any molecule that binds but thus inhibits the activity of GPR35 receptor. Examples include, but not limited to, CID2745687 (methyl-5-[(tert-butylcarbamothioylhydrazinylidene)methyl]-1-(2,4-difluorophenyl)-pyrazole-4-carboxylate); and ML145 (2-Hydroxy-4-[4-(5Z)-5-[(E)-2-methyl-3-phenylprop-2-enylidene]-4-oxo-2-sulfanylidene-1,3-thiazolidin-3-yl]butanoylamino]benzoic acid).

47. Higher, Increase, Elevate, Elevation

The terms “higher”, “increases”, “elevates”, or “elevation”, or like terms or variants of these terms, refer to increases above basal levels, e.g., as compared to a control. The terms “low”, “lower”, “reduces”, “decreases” “inhibit”, or “reduction”, or variation of these terms, refer to decreases below basal levels, e.g., as compared to a control. For example, basal levels are normal in vivo levels prior to, or in the absence of, or addition of an agent such as an agonist or antagonist to activity. For example, decreases or increases can be used to describe the binding of a molecule to a receptor. In this context, decreases would describe a situation of where the binding could be defined as having a K_{d} of 10⁻⁹ M, if this interaction decreased, meaning the binding lessened, the K_{d} could decrease to 10⁻⁶ M. It is understood that wherever one of these words is used it is also disclosed that it could be 1%, 5%, 10%, 20%, 50%, 100%, 500%, or 1000%, including intermediate values and ranges, increased or decreased from a control. This increase or decrease is typically in relation to some standard or expected value, in other words it is relative, but that it is not always necessary for the standard or relative value to be referred to. For example, “inhibits phosphorylation” means hindering or restraining the amount of phosphorylation that takes place relative to a standard or a control.

48. In the Presence of the Molecule

“in the presence of the molecule” or like terms refers to the contact or exposure of the cultured cell with the molecule. The contact or exposure can be taken place before, or at the time, the stimulus is brought to contact with the cell.

49. Index

An index or like terms is a collection of data. For example, an index can be a list, table, file, catalog, or like listing, that contains one or more modulation profiles. It is understood that an index can be produced from any combination of data. For example, a DMR profile can have a P-DMR, a N-DMR, and a RP-DMR. An index can be produced using the completed date of the profile, the P-DMR data, the N-DMR data, the RP-DMR data, or any point within these, or in combination of these or other data. The index is the collection of any such information. Typically, when comparing indexes, the indexes are of like data, i.e. P-DMR to P-DMR data.

50. Interact

“Interacts”, “interaction”, or the like, means that two (or more) molecules touch one another in a way beyond the touching that takes place because of random contacts between molecules. “Interacts” can be thought of as “binding” between two or more molecules, and therefore can have dissociation and association constants as well as equilibrium constants.

51. Inflammation

Inflammation is any specific or non-specific immune response. Inflammation is part of the complex biological response of vascular tissues to harmful stimuli, such as pathogens, damaged cells, or irritants. Inflammation is a protective attempt by the organism to remove the injurious stimuli and to initiate the healing process. Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

52. Known Molecule

A known molecule or like terms is a molecule with known pharmacological/biological/physiological/pathophysiological activity whose precise mode of action(s) may be known or unknown.

53. Known Modulator

A known modulator or like terms is a modulator where at least one of the targets is known with a known affinity. For example, a known modulator could be a GPR35 agonist, a GPR35 antagonist, etc.

54. Known GPR35 Agonist

A known GPR35 agonist is any GPR35 agonist that at the time it is used in an assay was known to be a GPR35 agonist, as shown in any way.

55. Known GPR35 Antagonist

A known GPR35 antagonist is any GPR35 antagonist that at the time it is used in an assay was known to be a GPR35 antagonist, as shown in any way. To date, there is a few of GPR35 antagonist reported in literature including CID2745687 (SPB05142) and ML145.

56. Metabolic Disorder

A metabolic disorder is a disorder of metabolism, such as diabetes, Type I diabetes, Type II diabetes, inadequate glucose tolerance, insulin resistance, hyperglycemia, hyperinsulinemia, hyperlipidemia, hypertriglyceridemia, hypercholesterolemia, dyslipidemia, obesity, premature or accelerated aging, Syndrome X, atherosclerosis, heart disease, stroke, hypertension and peripheral vascular disease.

57. Label-Free Biosensor Cellular Assay

A label free biosensor cellular assay or like terms is any assay that uses a label free biosensor to detect or monitor a cellular response.

58. Label

The terms label and tag as used herein refer to its presence as a moiety covalently or non-covalently bound to another residue such as the GPR35-hERG complex, wherein the label enables the location and or activity of the other residue to be monitored. In one example, the label can be fluorescent.

59. Ligand

A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity. Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

60. Library

A library or like terms is a collection. The library can be a collection of anything disclosed herein. For example, it can be a collection of indexes, an index library; it can be a collection of profiles, a profile library; or it can be a collection of DMR indexes, a DMR index library; Also, it can be a collection of molecules, a molecule library; it can be a collection of cells, a cell library; it can be a collection of markers, a marker library. A library can be, for example, random or non-random, determined or undetermined. For example, disclosed are libraries of DMR indexes or biosensor indexes of known modulators.

61. Maintaining

The word “maintaining” or like words refers to continuing a state. In the context of a treatment, maintaining can refer to, for example, less than 20%, 15%, 10%, 9%, 8%, 7%, 6%, 5%, 4%, 3%, 2%, 1%, or 0.1%, including intermediate values and ranges, change from a control, such a basal level, often a level in the absence of a treatment or in the presence of treatment with a placebo or standard.

62. Material

Material is the tangible part of something (chemical, biochemical, biological, or mixed) that goes into the makeup of a physical object.

63. Mimic

As used herein, “mimic” or like terms refers to performing one or more of the functions of a reference object. For example, a molecule mimic performs one or more of the functions of a molecule.

64. Modulate

To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000%, including intermediate values and ranges, increased from a control, or it could be 1%, 5%, 10%, 20%, 50%, or 100%, including intermediate values and ranges, decreased from a control.

65. Modulator

A modulator or like terms is a ligand that controls the activity of a cellular target. It is a signal modulating molecule binding to a cellular target, such as a target protein.

66. Modulate the Biosensor Signal of a Marker

“Modulate the biosensor signal of a marker” or like terms is to cause changes of the biosensor signal or profile of a cell in response to stimulation with a marker.

67. Modulate the DMR Signal

“Modulate the DMR signal” or like terms is to cause changes of the DMR signal or profile of a cell in response to stimulation with a marker.

68. Molecule

As used herein, the term “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

69. Molecule Mixture

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

70. Molecule-Treated Cell

A molecule-treated cell or like terms is a cell that has been exposed to a molecule.

71. Molecule Pharmacology

Molecule pharmacology or the like terms refers to the systems cell biology or systems cell pharmacology or mode(s) of action of a molecule acting on a cell. The molecule pharmacology is often characterized by, but not limited, toxicity, ability to influence specific cellular process(es) (e.g., proliferation, differentiation, reactive oxygen species signaling), or ability to modulate a specific cellular target.

72. Normal Individual Therapeutic Dose

In certain compositions, more than one active therapeutic agent is present. This is called a combination composition. Thus, within a combination composition, a normal individual therapeutic dose is the dosage that one of the active therapeutic agents is administered as a single active therapeutic agent.

73. Normalizing

Normalizing or like terms means, adjusting data, or a profile, or a response, for example, to remove at least one common variable. For example, if two responses are generated, one for a marker acting on a cell and one for a marker and molecule acting on the cell, normalizing would refer to the action of comparing the marker-induced response in the absence of the molecule and the response in the presence of the molecule, and removing the response due to the marker only, such that the normalized response would represent the response due to the modulation of the molecule against the marker. A modulation comparison is produced by normalizing a primary profile of the marker and a secondary profile of the marker in the presence of a molecule (modulation profile).

74. Optional

“Optional” or “optionally” means that the subsequently described event or circumstance may or may not occur, and that the description includes instances where said event or circumstance occurs and instances where it does not.

75. Or

The word “or” or like terms as used herein means any one member of a particular list and also includes any combination of members of that list.

76. Pathophysiologically Related to GPR35

Something is “pathophysiologically related to GPR35” if GPR35 is involved in the functional changes in a body associated with or resulting from disease or injury.

77. Pharmaceutically Acceptable

By “pharmaceutically acceptable”, it is meant a material that is not biologically or otherwise undesirable, i.e., the material can be administered to a subject without causing any undesirable biological effects or interacting in a deleterious manner with any of the other components of the pharmaceutical composition in which it is contained. A pharmaceutically acceptable carrier can be selected to minimize any degradation of the active ingredient and to minimize any adverse side effects in the subject, as would be well known to one of skill in the art. The carrier can be a solid, a liquid, or both, and can be formulated with the compound as a unit-dose composition, for example, a tablet, which can contain, for example, from 0.05% to 100%, from 0.05% to 99%, from 0.05% to 98%, from 0.05% to 97%, from 0.05% to 96%, from 0.05% to 95%, from 0.05% to 94%, from 0.05% to 93%, from 0.05% to 92%, from 0.05% to 91%, from 0.05% to 90%, from 0.05% to 85%, from 0.05% to 80%, from 0.05% to 75%, from 0.05% to 70%, from 0.05% to 65%, from 0.05% to 60%, from 0.05% to 55%, from 0.05% to 50%, from 0.05% to 45%, from 0.05% to 40%, from 0.05% to 35%, from 0.05% to 30%, from 0.05% to 25%, from 0.05% to 20%, from 0.05% to 15%, from 0.05% to 10%, from 0.05% to 5%, from 0.05% to 4%, from 0.05% to 3%, from 0.05% to 2%, from 0.05% to 1%, from 0.05% to 0.8%, from 0.05% to 0.6%, from 0.05% to 0.5%, from 0.05% to 0.4%, from 0.05% to 0.3%, from 0.05% to 0.2%, from 0.05% to 0.1%, from 0.1% to 100%, from 0.2% to 100%, from 0.3% to 100%, from 0.4% to 100%, from 0.5% to 100%, from 0.6% to 100%, from 0.8% to 100%, from 1% to 100%, from 2% to 100%, from 3% to 100%, from 4% to 100%, from 5% to 100%, from 10% to 100%, from 15% to 100%, from 20% to 100%, from 25% to 100%, from 30% to 100%, from 35% to 100%, from 40% to 100%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.8%, 0.6%, 0.5%, 0.4%, 0.3%, 0.2%, 0.1%, 0.05% by weight of the active compounds, including intermediate values and ranges. A disclosed compound can be coupled with suitable polymers as targetable drug carriers. Other pharmacologically active substances can also be present.

78. Positive Control

A “positive control” or like terms is a control that shows that the conditions for data collection can lead to data collection.

79. Potentiate

Potentiate, potentiated or like terms refers to an increase of a specific parameter of a biosensor response of a marker in a cell caused by a molecule. By comparing the primary profile of a marker with the secondary profile of the same marker in the same cell in the presence of a molecule, one can calculate the modulation of the marker-induced biosensor response of the cells by the molecule. A positive modulation means the molecule to cause increase in the biosensor signal induced by the marker.

80. Potency

Potency or like terms is a measure of molecule activity expressed in terms of the amount required to produce an effect of given intensity. For example, a highly potent drug evokes a larger response at low concentrations. The potency is proportional to affinity and efficacy. Affinity is the ability of the drug molecule to bind to a receptor.

81. Prodrug

“Prodrug” or the like terms refers to compounds that when metabolized in vivo, undergo conversion to compounds having the desired pharmacological activity. Prodrugs can be prepared by replacing appropriate functionalities present in pharmacologically active compounds with “pro-moieties” as described, for example, in H. Bundgaar, Design of Prodrugs (1985). Examples of prodrugs include ester, ether or amide derivatives of the compounds herein, and their pharmaceutically acceptable salts. For further discussions of prodrugs, see e.g., T. Higuchi and V. Stella “Pro-drugs as Novel Delivery Systems,” ACS Symposium Series 14 (1975) and E. B. Roche, ed., Bioreversible Carriers in Drug Design (1987).

82. Publications

Throughout this application, various publications are referenced. The disclosures of these publications in their entireties are hereby incorporated by reference into this application to more fully describe the state of the art to which this pertains. The references disclosed are also individually and specifically incorporated by reference herein for the material contained in them that is discussed in the sentence in which the reference is relied upon.

83. Ranges

Ranges can be expressed herein as from “about” one particular value, to “about” another particular value. When such a range is expressed, some forms includes from the one particular value to the other particular value. Similarly, when values are expressed as approximations, by use of the antecedent “about,” the particular value constitutes one of the encompassed values. The endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. Throughout the application, data are provided in a number of different formats, and that these data represent endpoints and starting points, and ranges for any combination of the data points. For example, if a particular datum point “10” and a particular datum point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

84. Receptor

A receptor or like terms is a protein molecule embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

85. Reduce

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

86. Reducing the Risk of

“Reducing the risk of” refers to lowering the chance of an event or characteristic to happen. For example, reducing the risk of disease means lowering the chance that disease will occur, for example, in a subject.

87. “Robust Biosensor Signal”

A “robust biosensor signal” is a biosensor signal whose amplitude(s) is significantly (such as 3×, 10×, 20×, 100×, or 1000×) above either the noise level, or the negative control response. The negative control response is often the biosensor response of cells after addition of the assay buffer solution (i.e., the vehicle). The noise level is the biosensor signal of cells without further addition of any solution. The cells are preferably always covered with a solution before addition of any solution.

88. “Robust DMR Signal”

A “robust DMR signal” or like terms is a DMR form of a “robust biosensor signal.”

89. Response

A response or like terms is any reaction to any stimulation.

90. Sample

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

91. Salt(s) and Pharmaceutically Acceptable Salt(s)

The compounds of this disclosure may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of Formula (I), (II), or (III), with an acid whose anion, or a base whose cation, is generally considered suitable for human consumption. Pharmaceutically acceptable salts are particularly useful as products of the methods of the present disclosure because of their greater aqueous solubility relative to the parent compound. For use in medicine, the salts of the compounds of this disclosure are non-toxic “pharmaceutically acceptable salts.” Salts encompassed within the term “pharmaceutically acceptable salts” refer to non-toxic salts of the compounds of this disclosure which are generally prepared by reacting the free base with a suitable organic or inorganic acid.

Suitable pharmaceutically acceptable acid addition salts of the compounds of the present disclosure when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate. Furthermore, where the compounds of the disclosure carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In embodiments, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary, or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (CrC₆) halides (e.g., methyl, ethyl, propyl, and butyl chlorides, bromides, and iodides), dialkyl sulfates (i.e., dimethyl, diethyl, dibuytl, and diamyl sulfates), long chain halides (i.e., decyl, lauryl, myristyl, and stearyl chlorides, bromides, and iodides), arylalkyl halides (i.e., benzyl and phenethyl bromides), and others.

In embodiments, hemisalts of acids and bases may also be formed, for example, hemisulphate and hemicalcium salts.

The compounds of the disclosure and their salts may exist in both unsolvated and solvated forms.

92. Signaling Pathway(s)

A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

93. Subject

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

94. Solvate

The compounds herein, and the pharmaceutically acceptable salts thereof, may exist in a continuum of solid states ranging from fully amorphous to fully crystalline. They may also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, d₆-acetone, d₆-DMSO).

A currently accepted classification system for solvates and hydrates of organic compounds is one that distinguishes between isolated site, channel, and metal-ion coordinated solvates and hydrates. See, e.g., K. R. Morris (H. G. Brittain, ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm.

The compounds herein, and the pharmaceutically acceptable salts thereof, may also exist as multi-component complexes (other than salts and solvates) in which the compound and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together (see, e.g., O. Almarsson and M. J. Zaworotko, Chem. Commun., 17:1889-1896 (2004)). For a general review of multi-component complexes, see J. K. Haleblian, J. Pharm. Sci., 64(8):1269-88 (1975).

95. Stable

When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

96. Substance

A substance or like terms is any physical object. A material is a substance. Molecules, ligands, markers, cells, proteins, and DNA can be considered substances. A machine or an article would be considered to be made of substances, rather than considered a substance themselves.

97. Test Molecule

A test molecule or like terms is a molecule which is used in a method to gain some information about the test molecule. A test molecule can be an unknown or a known molecule.

98. Tissue

Tissue or like terms refers to a collection of cells. Typically a tissue is obtained from a subject.

99. Treating

By “treating” or “treatment” is meant the medical management of a patient with the intent to cure, ameliorate, stabilize, or prevent a disease, pathological condition, or disorder. These terms include active treatment, that is, treatment directed specifically toward the improvement of a disease, pathological condition, or disorder, and also includes causal treatment, that is, treatment directed toward removal of the cause of the associated disease, pathological condition, or disorder. These terms can mean that the symptoms of the underlying disease are reduced, or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. In certain situations a treatment can inadvertently cause harm. In addition, these terms include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. These terms mean both treatment having a curing or alleviating purpose and treatment having a preventive purpose. The treatment can be made either acutely or chronically. Treatment can mean a reduction or one or more symptoms or characteristics by at least 10%, 20%, 30%, 40%, 50%, 60%, 70%, 80%, 90%, 95%, 99%, 99.9%, 99.99%, 100%, including intermediate values and ranges, relative to a control. In the context of these terms, preventing refers to the ability of a compound or composition (such as the disclosed compounds and compositions) to prevent a disease identified herein in patients diagnosed as having the disease or who are at risk of developing such disease. In this context, preventing includes the delaying the onset of the disease relative to a control. These terms do not require that the treatment in fact be effective to produce any of the intended results. It is enough that the results are intended.

100. Therapeutic Agent

A therapeutic agent or like term is any molecule or composition in which the molecule or composition is useful in preventing or treating conditions or diseases within the therapeutic field. For example, anti-cancer agents can be any agent that can prevent the formation of cancer cell in a subject, reduce the number of cancer cells in a subject, or eliminate some or all cancer cells in a subject.

101. Therapeutically Effective

The term “therapeutically effective” means that the amount of the composition used is of sufficient quantity to treat a subject as defined herein.

102. Toxicity

Toxicity is the degree to which a substance or molecule is able to damage something, such as a cell, a tissue, an organ, or a whole organism, that has been exposed to the substance or molecule. For example, the liver, or cells in the liver, hepatocytes, can be damaged by certain substances.

103. Toxicity Marker

A toxicity marker is any reagent, molecule, substance, etc., that can be used for identifying, diagnosing, prognosing a level of toxicity of a substance, in for example, an organism or cell or tissue or organ.

104. Transactivate

“Transactivate” refers to the process that the activation of a receptor in a cell can also activate another receptor in the same cell. Such transactivation can be direct (i.e., both receptors form a complex such as dimer or oligomer, such that the activation of the 1^{st} receptor cause a conformational change in the 2^{nd} receptor, thus leading to the activation of the 2^{nd} receptor) or indirect (i.e., the two receptors are not necessarily within a signaling complex; however, the activation of 1^{st} receptor leads to a pathway in which a signaling protein within the pathway activates the 2^{nd} receptor).

105. Trigger

A trigger or like terms refers to the act of setting off or initiating an event, such as a response.

106. Values

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein, including intermediate values and ranges.

Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

107. Unknown Molecule

An unknown molecule or like terms is a molecule with unknown biological, pharmacological, physiological, pathophysiological, or like activity.

108. Weight %

References in the specification and concluding claims to parts by weight, of a particular element or component in a composition or article, denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

### C. METHODS

In embodiments, the disclosure provides methods related to modulation of GPR35 activity. In embodiments, modulation of GPR35 activity treats disease in a subject. In embodiments, modulation of GPR35 activity reduces the risk of disease in a subject. In embodiments, modulation of GPR35 activity reduces the risk of or treats disease in a subject. In embodiments, the disease is pathophysiologically related to GPR35 activity.

In embodiments, modulation of GPR35 activity reduces GPR35 activity. In embodiments, modulation of GPR35 activity increases GPR35 activity.

In embodiments, the disclosure provides methods of reducing the risk of disease, treating disease, or both, in a subject, including, for example:

administering to the subject a therapeutically effective amount of a compound of the Formula (I):

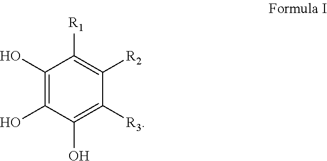

In embodiments, R¹, R², and R³ can independently be, for example, —H, —NO₂, —COOH, halide, acyl halide, substituted or unsubstituted alkyl, substituted or unsubstituted —NH-alkyl, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, or a substituted or unsubstituted alkoxy.

In embodiments, at least one, two, or three of R¹, R² and R³ are not —H. In embodiments at least one of R¹, R² and R³ are not —H. In embodiments, at least two of R¹, R² and R³ are not —H. In embodiments, R¹, R² and R³ are not —H.

In embodiments, R¹ and R² optionally together form a cyclic moiety. In embodiments, R¹ and R² optionally together form a substituted or unsubstituted aryl, substituted or unsubstituted heteroaryl, substituted or unsubstituted cycloalkenyl or substituted or unsubstituted heterocyclyl. In embodiments, R¹ and R² optionally together form a substituted heterocyclyl.

In embodiments, the disclosure provides methods of reducing the risk of disease, treating disease, or both, in a subject, including, for example:

administering to the subject a therapeutically effective amount of a compound of the Formula (II):

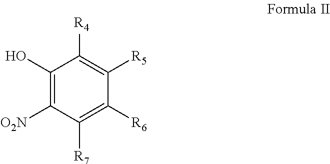

In embodiments R⁴, R⁵, R⁶, and R⁷ can independently be, for example, —H, —OH, halide, acyl halide, substituted or unsubstituted alkyl, substituted or unsubstituted —NH-alkyl, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, or a substituted or unsubstituted alkoxy. In embodiments, R⁷ can be, for example, —H, —OH, —COOH, or halide.

In embodiments, R⁴, R⁵, R⁶, and R⁷ can independently be, for example, each individually —H, —OH, halide, acyl halide, substituted or unsubstituted alkyl, substituted or unsubstituted —NH-alkyl, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, or a substituted or unsubstituted alkoxy.

In embodiments, R⁶ can be, for example, —H, —NO₂, substituted or unsubstituted alkyl, substituted or unsubstituted alkenyl or substituted or unsubstituted aryl. In embodiments, R⁶ can be, for example, substituted or unsubstituted aryl. In embodiments, R⁶ can be, for example, a substituted or unsubstituted alkyl, or a substituted or unsubstituted alkenyl.

In embodiments, R⁶ and R⁷ can optionally together form, for example, a substituted or unsubstituted aryl, substituted or unsubstituted heteroaryl, substituted or unsubstituted cycloalkenyl or substituted or unsubstituted heterocyclyl. In embodiments, R⁶ and R⁷ can optionally together form, for example, a substituted or unsubstituted aryl (Ar). In embodiments, R⁵ and R⁶ can optionally together form, for example, a substituted or unsubstituted aryl.

In embodiments, the disclosure provides methods of reducing the risk of or treating disease in a subject, including administering to the subject a therapeutically effective amount of a compound having Formula (III):

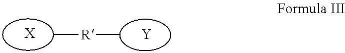

In embodiments, X and Y can be identical or different. X and Y independently can have a structure of the formula (V) to (IX):

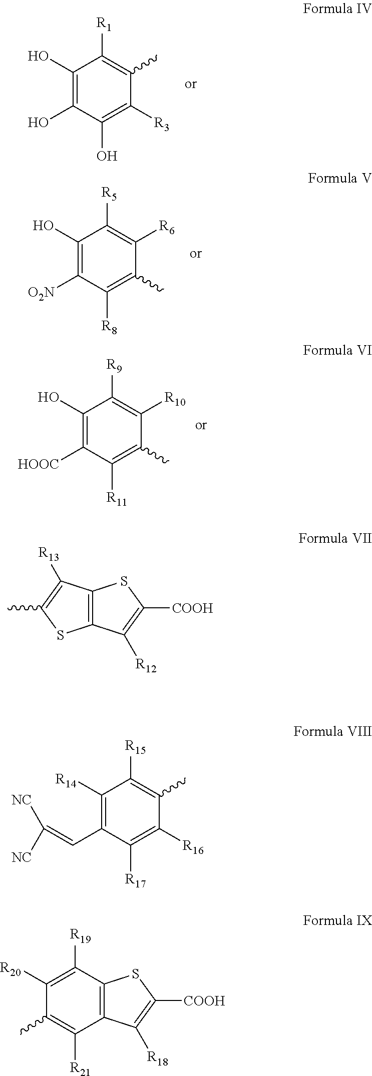

In embodiments in any of the Formulas (IV), (V), (VI), (VII), (VIII), or (IX), R¹, R³, R⁵, R⁶, R⁸, R⁹, R¹⁰, R¹¹, R¹², R¹³, R¹⁶, R¹⁷, R¹⁸, R¹⁹, R²⁰, and R²¹, can each individually and independently be, for example: —H, —OH, halide, acyl halide, substituted or unsubstituted alkyl, substituted or unsubstituted —NH-alkyl, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, or a substituted or unsubstituted alkoxy.

In embodiments, R′ can be, for example, a single covalent carbon-carbon bond, —N═N—, —NH—NH—, —O—, —(CH₂CH₂O)ₙ—, substituted or unsubstituted alkyl, substituted or unsubstituted —NH-alkyl, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, or a substituted or unsubstituted alkoxy.

In embodiments, a specific structure of a compound of Formula (I) can be, for example:

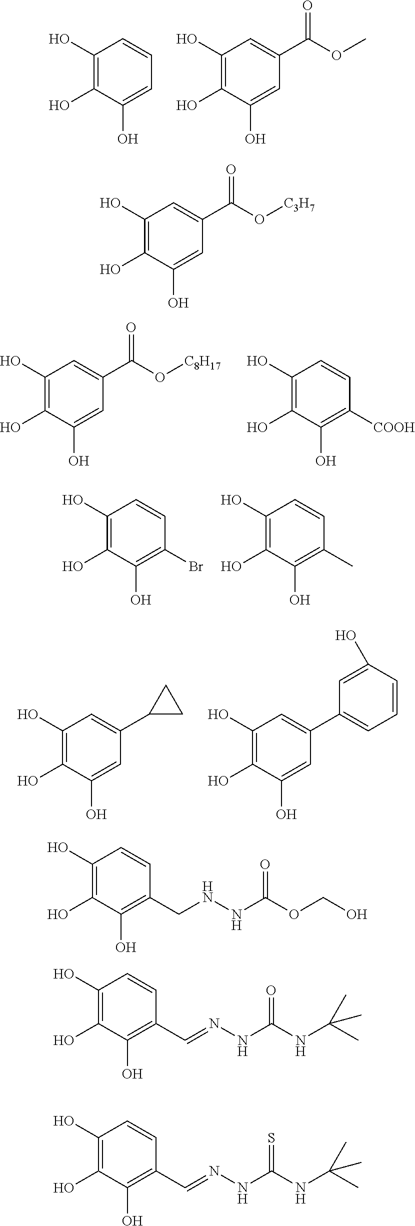

In embodiments, a specific structure of a compound of Formula (II) can be, for example:

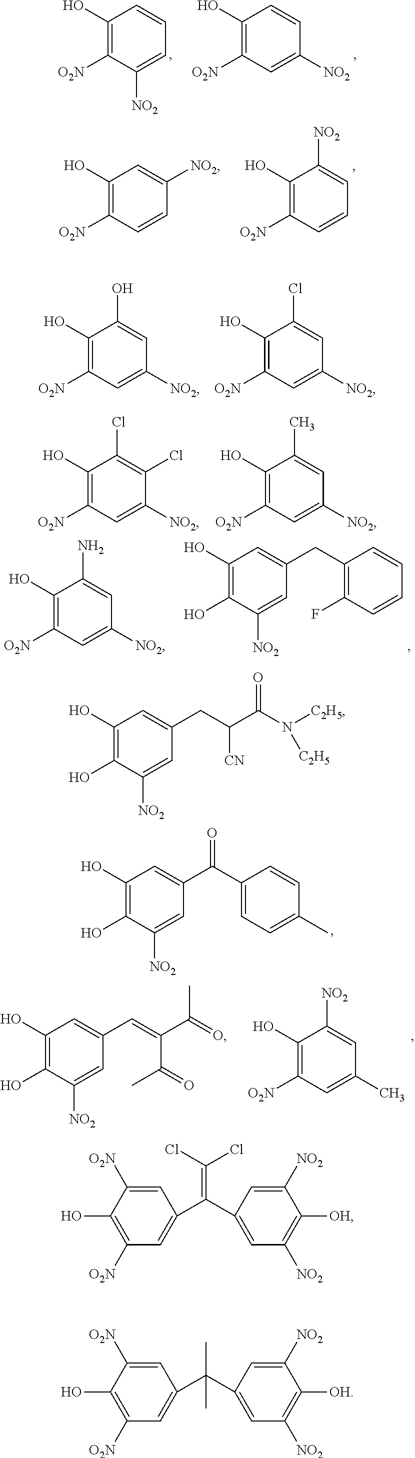

In embodiments, a specific structure of a compound of Formula (III) can be, for example:

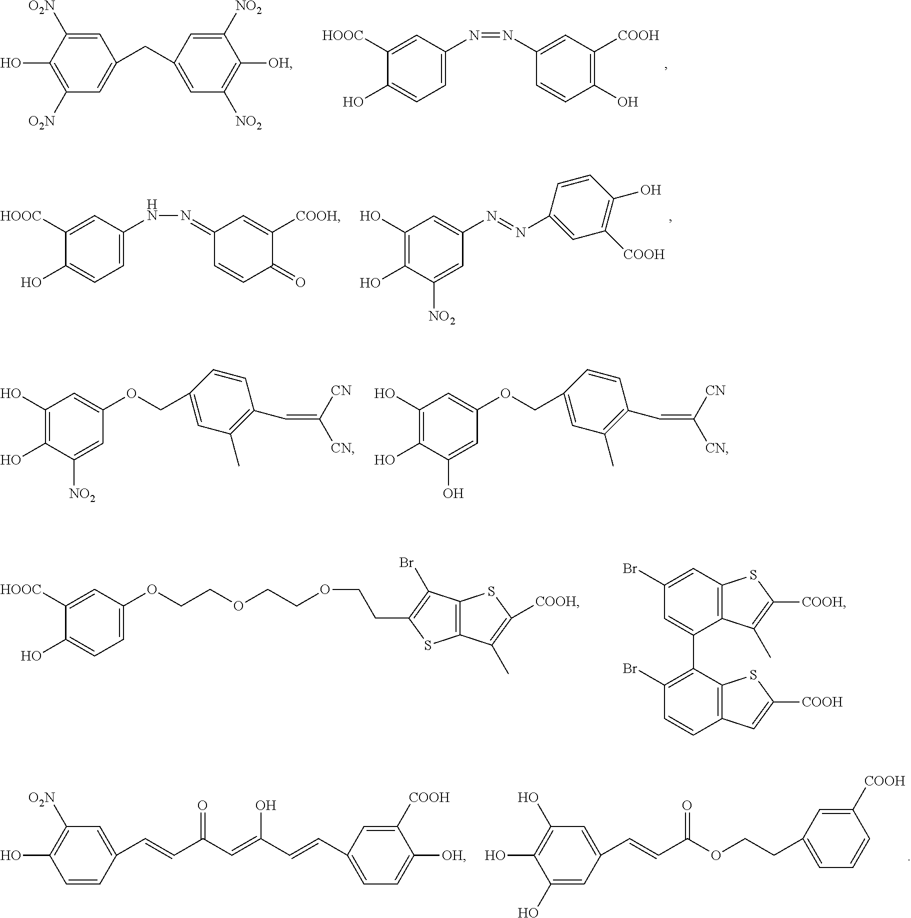

In embodiments, compounds of the a compound of the Formula (I), (II), or (III), can be in the form of a pharmaceutically acceptable salt, solvate, clathrate, or a prodrug thereof. In some forms the pharmaceutically acceptable salt can be metal chelating forms. In some forms the pharmaceutically acceptable salt can be Cu²⁺, or Zn²⁺ chelating forms. In embodiments, the pharmaceutically acceptable salt for compounds having a —COOH group can be, for example, Cu(II) or Zn(II) chelating forms.

In embodiments, the disease can be pathophysiologically related to GPR35 activity. In embodiments, Formula (I), (II), or (III) can be in the form of a pharmaceutically acceptable salt, solvate, clathrate or prodrug thereof and the disease can be pathophysiologically related to GPR35 activity.

In embodiments, Formula (I), (II), or (III), or a pharmaceutically acceptable salt, solvate, clathrate or prodrug thereof, can be a GPR35 modulator. In embodiments, the GPR35 modulator can be a GPR35 agonist. In embodiments, the GPR35 modulator can be a GPR35 antagonist.

In embodiments, the disease can be selected from the group consisting of inflammation, metabolic disorder, congestive heart failure, and cancer. In embodiments, the disease can be a metabolic disorder. In embodiments, the metabolic disorder can be selected from the group consisting of diabetes, Type I diabetes, Type II diabetes, inadequate glucose tolerance, insulin resistance, hyperglycemia, hyperinsulinemia, hyperlipidemia, hypertriglyceridemia, hypercholesterolemia, dyslipidemia, obesity, aging, Syndrome X, atherosclerosis, heart disease, stroke, hypertension and peripheral vascular disease. In embodiments, the disease can be cancer. In embodiments, the cancer can be selected from the group consisting of prostate cancer, leukemia, hormone dependent cancers, breast cancer, colon cancer, lung cancer, epidermal cancer, liver cancer, esophageal cancer, stomach cancer, cancer of the brain, and cancer of the kidney.

In embodiments, compounds of the Formula (I), (II), (III), or a pharmaceutically acceptable salt thereof, can be administered by one or more routes selected from a group consisting of rectal, buccal, sublingual, intravenous, subcutaneous, intradermal, transdermal, intraperitoneal, oral, eye drops, parenteral and topical administration. In embodiments, compounds of the Formula (I), (II), (III), or a pharmaceutically acceptable salt thereof, can be administered orally. In embodiments, compounds of the Formula (I), (II), (III), or a pharmaceutically acceptable salt thereof, can be administered subcutaneously. In embodiments, compounds of the Formula (I), (II), (III), or a pharmaceutically acceptable salt thereof, can be administered rectally. In embodiments, compounds of the Formula (I), (II), (III), or a pharmaceutically acceptable salt thereof, can be administered topically.

In embodiments, the subject can be diagnosed with a disease pathophysiologically related to GPR35 activity. In embodiments, the subject can be in need of a drug for a disease pathophysiologically related to GPR35 activity. In embodiments, the subject can be administered a therapeutically effective amount of a compound of the Formula (I), (II), or (III). In embodiments, the subject is at risk of having a disease pathophysiologically related to GPR35 activity.

Also disclosed herein are pharmaceutical compositions including a compound of the Formula (I), (II), or (III), and mixtures thereof.

In embodiments, a specific structure of a compound of the Formula (III) can be, for example:

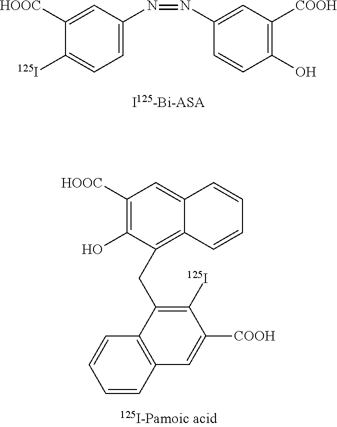 [SMILES: O=C(O)c1cc(/N=N\c2ccc([125I])c(C(=O)O)c2)ccc1O ; O=C(O)c1cc2ccccc2c(Cc2c([125I])c(C(=O)O)cc3ccccc23)c1O]

Both I¹²⁵-Bi-ASA and ¹²⁵I-pamoic acid are radioactive and can be used for measuring the binding of a known or unknown ligands binding to GPR35 receptors in vitro or in vivo.

1. Administration

The disclosed compounds can be administered by any suitable route, preferably in the form of a pharmaceutical composition adapted to such a route, and in a dose effective for the treatment or prevention intended. The active compounds and compositions, for example, can be administered orally, rectally, parenterally, occularly, inhalationally, or topically.

Oral administration of a solid dose form can be, for example, presented in discrete units, such as hard or soft capsules, pills, cachets, lozenges, or tablets, each containing a predetermined amount of at least one of the disclosed compound or compositions. In some forms, the oral administration can be in a powder or granule form. In some forms, the oral dose form is sub-lingual, such as, for example, a lozenge. In such solid dosage forms, the compounds of Formula (I) are ordinarily combined with one or more adjuvants. Such capsules or tablets can contain a controlled-release formulation. In the case of capsules, tablets, and pills, the dosage forms also can comprise buffering agents or can be prepared with enteric coatings.

In some forms, oral administration can be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also can comprise adjuvants, such as wetting, emulsifying, suspending, flavoring (e.g., sweetening), or perfuming agents.

In some forms, the disclosed compositions can comprise a parenteral dose form. “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) can be formulated according to the known art using suitable dispersing, wetting agents, or suspending agents.

In some forms, the disclosed compositions can include a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation can include a compound which enhances absorption or penetration of the active ingredient through the skin or other affected areas. When the compounds and compositions are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes can also be used. Typical carriers include alcohol, water, mineral oil, liquid petrolatum, white petrolatum, glycerin, polyethylene glycol and propylene glycol. Penetration enhancers can be incorporated, see, for example, J Pharm Sci, 88 (10), 955-958, by Finnin and Morgan (October 1999).

Formulations suitable for topical administration to the eye include, for example, eye drops wherein the disclosed compound or composition is dissolved or suspended in suitable carrier. A typical formulation suitable for ocular or aural administration can be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, can be incorporated together with a preservative, such as benzalkonium chloride. Such formulations can also be delivered by iontophoresis.

For intranasal administration or administration by inhalation, the active disclosed compounds are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder can comprise a bioadhesive agent, for example, chitosan or cyclodextrin.

In some forms, the disclosed compositions can comprise a rectal dose form. Such rectal dose form can be in the form of, for example, a suppository. Cocoa butter is a traditional suppository base, but various alternatives can be used as appropriate.

Other carrier materials and modes of administration known in the pharmaceutical art can also be used. The disclosed pharmaceutical compositions can be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

The disclosed compounds of the Formula (I), (II), or (III), can be used, alone or in combination with other therapeutic agents, in the treatment or reduction of risk of various conditions or disease states. The disclosed compound(s) and composition(s) of the Formulas (I), (II), or (III), and other therapeutic agent(s) can be administered simultaneously (either in the same dosage form or in separate dosage forms) or sequentially. An exemplary therapeutic agent can be, for example, one selected from the group consisting of anti-inflammation agent, anti-metabolic-disorder agent, anti-congestive-heart-failure agent, anti-cancer agent, kynurenic acid, and a compound of the Formula (I), (II), or (III). In embodiments, an anti-cancer agent and a compound of the Formula (I), (II), (III), can be administered together or in combination.

The administration of two or more compounds “in combination” means that the two compounds are administered closely enough in time that the presence of one alters the biological effects of the other. The two or more compounds can be administered simultaneously, concurrently or sequentially. Additionally, simultaneous administration can be carried out by mixing the compounds prior to administration or by administering the compounds at the same point in time but at different anatomic sites or using different routes of administration. The phrases “concurrent administration,” “co-administration,” “simultaneous administration,” and “administered simultaneously” mean that the compounds are administered in combination.

The dosage regimen for the compounds or compositions containing the compounds can be based on a variety of factors, including the type, age, weight, sex and medical condition of the patient; the severity of the condition; the route of administration; and the activity of the particular compound employed. Thus the dosage regimen can vary widely. Dosage levels of the order from about 0.001 mg to about 100 mg per kilogram of body weight per day are useful in the treatment or prevention of the above-indicated conditions. Other effective dosages regimens of a disclosed compounds (administered in single or divided doses) include but are not limited to: from about 0.01 to about 100 mg/kg/day, from about 0.1 to about 50 mg/kg/day, from about 0.5 to about 30 mg/kg/day, from about 0.01 to about 10 mg/kg/day, and from about 0.1 to about 1.0 mg/kg/day. Dosage unit compositions can contain such amounts or submultiples thereof to make up the daily dose. In many instances, the administration of the compound will be repeated a plurality of times in a day. Multiple doses per day typically can be used to increase the total daily dose, if desired.

For oral administration, the compositions can be provided in the form of tablets containing, for example, 0.01, 0.05, 0.1, 0.5, 1.0, 2.5, 5.0, 10.0, 15.0, 25.0, 50.0, 75.0, 100, 125, 150, 175, 200, 250, or 500 milligrams of the active ingredient for the symptomatic adjustment of the dosage to the patient. A medicament typically contains from about 0.01 mg to about 500 mg of the active ingredient, or from about 1 mg to about 100 mg of active ingredient. Intravenously, doses can range from about 0.1 to about 10 mg/kg/minute during a constant rate infusion.

In embodiments, the disclosure provides pharmaceutical compositions comprising an effective amount of a compound of the disclosure or a pharmaceutically accepted salt, solvate, clathrate, or prodrug thereof; and a pharmaceutically acceptable carrier or vehicle. These compositions can further comprise additional agents. These compositions are useful for modulating the activity of GPR35, thus to improve the prevention and treatment of GPR35 associated human diseases such as metabolic disorders.

The compounds or compositions described in the methods herein can be administered adjunctively with other active compounds. These compounds include but are not limited to analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics. “Adjunctive administration”, as used herein, means the compounds or compositions can be administered in the same dosage form or in separate dosage forms with one or more other active agents.

Specific examples of compounds that can be adjunctively administered with a compound of the Formula (I), (II), or (III), can include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenyloin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

2. Controlled Release Formulations

The pharmaceutical compositions described herein can be formulated for controlled release including immediate release, delayed release, extended release, pulsatile release, and combinations thereof.

i. Nano- and Microparticles

For parenteral administration, the one or more pharmaceutical compositions having a compound of the Formula (I), (II), or (III), and optionally one or more additional active agents, can be incorporated into microparticles, nanoparticles, or combinations thereof that provide controlled release of the pharmaceutical compositions having a compound of the Formula (I), (II), or (III), and one or more additional active agents. In embodiments wherein the formulations contains two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, compounds of the Formula (I), (II), or (III), and one or more additional active agents can be incorporated into polymeric microparticles which provide controlled release of the drug(s). Release of the drug(s) is controlled by diffusion of the drug(s) out of the microparticles or degradation of the polymeric particles by hydrolysis or enzymatic degradation. Suitable polymers include ethylcellulose and other natural or synthetic cellulose derivatives.

Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

In some situations, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins which are water insoluble, such as zein, can also be used as materials for the formation of drug containing microparticles. Additionally, proteins, polysaccharides and combinations thereof which are water soluble can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In embodiments, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In embodiments, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water soluble protein can be spray coated onto the microparticles and subsequently cross-linked by the one of the methods described above. Alternatively, drug containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

In embodiments, it may be desirable to provide continuous delivery of one or more pharmaceutical compositions having a compound of the Formula (I), (II), or (III), to a patient in need thereof. For intravenous or intraarterial routes, this can be accomplished using drip systems, such as by intravenous administration. For topical applications, repeated application can be done or a patch can be used to provide continuous administration of the pharmaceutical compositions having a compound of the Formula (I), (II), or (III), over an extended period of time.

ii. Injectable/Implantable Solid Implants

The pharmaceutical compositions having a compound of the Formula (I), (II), or (III), can be incorporated into injectable/implantable solid or semi-solid implants, such as polymeric implants. In embodiments, the pharmaceutical compositions having a compound of the Formula (I), (II), or (III), are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the pharmaceutical compositions having a compound of the Formula (I), (II), or (III), can be incorporated into a polymer matrix and molded, compressed, or extruded into a device that is a solid at room temperature. For example, the pharmaceutical compositions having a compound of the Formula (I), (II), or (III), can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof, and compressed into solid device, such as disks, or extruded into a device, such as rods.

The release of the one or more pharmaceutical compositions having a compound of the Formula (I), (II), or (III), from the implant can be varied by selection of the polymer, the molecular weight of the polymer, or modification of the polymer to increase degradation, such as the formation of pores or incorporation of hydrolyzable linkages. Methods for modifying the properties of biodegradable polymers to vary the release profile of the pharmaceutical compositions having a compound of the Formula (I), (II), or (III), from the implant are well known in the art.

iii. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition which may include plasticizers, pigments, colorants, stabilizing agents, and glidants. Delayed release dosage formulations may be prepared as described in standard references. These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders can be used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid, and polyvinylpyrrolidone.

Lubricants can be used to facilitate tablet manufacture. Examples of suitable lubricants include, but are not limited to, magnesium stearate, calcium stearate, stearic acid, glycerol behenate, polyethylene glycol, talc, and mineral oil.

Disintegrants can be used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

a. Controlled Release Formulations

Oral dosage forms, such as capsules, tablets, solutions, and suspensions, can be for formulated for controlled release. For example, the one or more pharmaceutical compositions having a compound of the Formula (I), (II), or (III), and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In embodiments, the one or more pharmaceutical compositions having a compound of the Formula (I), (II), or (III), and optionally one or more additional active agents are dispersed in a matrix material, which gels or emulsifies upon contact with an aqueous medium, such as physiological fluids. In the case of gels, the matrix swells entrapping the active agents, which are released slowly over time by diffusion, degradation, or both of the matrix material. Such matrices can be formulated as tablets or as fill materials for hard and soft capsules.

In embodiments, the one or more pharmaceutical compositions having a compound of the Formula (I), (II), or (III), and optional one or more additional active agents can be formulated into a solid oral dosage form, such as a tablet or capsule, and the solid dosage form is coated with one or more controlled release coatings, such as a delayed release coatings or extended release coatings. The coating or coatings may also contain the pharmaceutical compositions having a compound of the Formula (I), (II), or (III), and additional active agents.

(A) Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In embodiments, the plastic material can be a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid) (anhydride), polymethacrylate, polyacrylamide, poly(methacrylic acid anhydride), and glycidyl methacrylate copolymers.

In embodiments, the acrylic polymer can be comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In embodiments, the acrylic polymer can be an acrylic resin lacquer such as that which is commercially available from Rohm Pharma under the tradename Eudragit®. In embodiments, the acrylic polymer can be a mixture of two acrylic resin lacquers commercially available from Rohm Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit®L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above, such as Eudragit® RL/RS, may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit®RL and 50% Eudragit® RS, and 10% Eudragit® RL and 90% Eudragit®RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant can be necessary in a tablet formulation to prevent the tablet and punches from sticking in the die. The lubricant can be chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

(B) Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

iv. Topical Formulations

Suitable dosage forms for topical administration include creams, ointments, salves, sprays, gels, lotions, emulsions, and transdermal patches. The formulation may be formulated for transmucosal, transepithelial, transendothelial, or transdermal administration. The compounds can also be formulated for intranasal delivery, pulmonary delivery, or inhalation. The compositions may further contain one or more chemical penetration enhancers, membrane permeability agents, membrane transport agents, emollients, surfactants, stabilizers, and combination thereof.

a. Topical Formulations

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In embodiments, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In embodiments, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In embodiments, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

(A) Lotions, Creams, Gels, Ointments, Emulsions, and Foams

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In embodiments, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In embodiments, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In embodiments, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In embodiments, it may be desirable to provide continuous delivery of one or more pharmaceutical compositions having a compound of the Formula (I), (II), or (III), to a patient in need thereof. For topical applications, repeated application can be done or a patch can be used to provide continuous administration of the pharmaceutical compositions having a compound of the Formula (I), (II), or (III), over an extended period of time.

v. Pulmonary Formulations

In embodiments, the pharmaceutical compositions having a compound of the Formula (I), (II), or (III), are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids.

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung. The deep lung, or alveoli, are the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal or upper respiratory administration.

Preferably, the aqueous solutions is water, physiologically acceptable aqueous solutions containing salts or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In embodiments solvents that are low toxicity organic (i.e., nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the pharmaceutical compositions having a compound of the Formula (I), (II), or (III). An appropriate solvent should be used that dissolves the pharmaceutical compositions having a compound of the Formula (I), (II), or (III), or forms a suspension of the pharmaceutical compositions having a compound of the Formula (I), (II), or (III). The solvent should be sufficiently volatile to enable formation of an aerosol of the solution or suspension. Additional solvents or aerosolizing agents, such as freons, can be added as desired to increase the volatility of the solution or suspension.

In embodiments, compositions may contain minor amounts of polymers, surfactants, or other excipients well known to those of the art. In this context, “minor amounts” means no excipients are present that might affect or mediate uptake of the pharmaceutical compositions having a compound of the Formula (I), (II), or (III), in the lungs and that the excipients that are present are present in amount that do not adversely affect uptake of pharmaceutical compositions having a compound of the Formula (I), (II), or (III), in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different EGS may be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In embodiments, the liposome can include a ligand molecule specific for a receptor on the surface of the target cell to direct the liposome to the target cell.

### D. EXAMPLES

Various embodiments will be further clarified by the following examples.

### 1. Materials and Methods

i. Materials

Zaprinast was obtained from BioMol International Inc (Plymouth Meeting, Pa.). Epic® 384 biosensor microplates cell culture compatible were obtained from Corning Inc. (Corning, N.Y.). Compounds with a purity greater than 95% were purchased from various vendors or custom synthesized. CID2745687 (SPB05142) was obtained from Ryan Scientific, Inc. (Mt. Pleasant, S.C.). ML145 was obtained from Tocris Biosciences Co. (St. Louis, Mo.).

HT-29 cells were obtained from American Type Cell Culture (Manassas, Va.). The cell culture medium was McCoy's 5a Medium Modified supplemented with 10% FBS, 4.5 g/liter glucose, 2 mM glutamine, and antibiotics.

Tango™ GPR35-bla U2OS cells were purchased from Invitrogen. The cells were cultured according to the protocols recommended by the supplier. Briefly, the cells were passed using McCoy's 5A medium (Invitrogen 16600-082) supplemented with 10% dialyzed fetal bovine serum, 0.1 μM NEAA, 25 μM Hepes (pH 7.3), 1 mM sodium pyruvate, 100 U/ml penicillin, 100 μg/ml streptomycin, 200 μg/ml zeocin, 50 μg/ml hygromycin, and 100 μg/ml geneticin in a humidified 37° C./5% CO₂ incubator.

ii. Optical Biosensor System and Cell Assays

Epic® beta version wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of about 15 sec. Also Epic® commercial systems were used, wherein a liquid handler accessory was attached to Epic® reader system.

The RWG biosensor is capable of detecting minute changes in local index of refraction near the sensor surface. Since the local index of refraction within a cell is a function of density and its distribution of biomass (e.g., proteins, molecular complexes), the biosensor exploits its evanescent wave to non-invasively detect ligand-induced dynamic mass redistribution in native cells. The evanescent wave extends into the cells and exponentially decays over distance, leading to a characteristic sensing volume having a height of about 150 nm, implying that any optical response mediated through the receptor activation only represents an average over the portion of the cell that the evanescent wave is sampling. The aggregation of many cellular events downstream the receptor activation determines the kinetics and amplitudes of a ligand-induced DMR.

For biosensor cellular assays, cells were typically grown using about 1 to 2×10⁴ cells per well at passage 3 to 15 suspended in 50 μl of the corresponding culture medium in the biosensor microplate, and were cultured at 37° C. under air/5% CO₂ for about 1 day. The confluency for all cells at the time of assays was about 95% to 100%. The molecule solutions were made by diluting the stored concentrated solutions with the HBSS (1× Hanks balanced salt solution, plus 20 mM Hepes, pH 7.1), and transferred into a 384well polypropylene molecule storage plate to prepare a molecule source plate. Both molecule and marker source plates were made separately when a two-step assay was performed. In parallel, the cells were washed twice with the HBSS and maintained in 30 μl of the HBSS to prepare a cell assay plate. Both the cell assay plate and the molecule and marker source plate(s) were then incubated in the hotel of the reader system. After about 1 hr of incubation the baseline wavelengths of all biosensors in the cell assay microplate were recorded and normalized to zero. Afterwards, a 2 to 10 minute continuous recording was carried out to establish a baseline, and to ensure that the cells reached a steady state. Cellular responses were then triggered by pipetting 10 μl of the marker solutions into the cell assay plate using the on-board liquid handler.

To study the influence of molecules on a marker-induced response, a second stimulation with the marker at a fixed dose (typically at EC80 or EC100) was applied. The resonant wavelengths of all biosensors in the microplate were normalized again to establish a second baseline, right before the second stimulation. The two stimulations were usually separated by about 1 hr.

All studies were carried out at a controlled temperature (28° C.). At least two independent sets of experiments, each with at least three replicates, were performed. The assay coefficient of variation was found to be <10%.

iii. Tango β-Arrestin Translocation Gene Reporter Assays

Tango™ GPR35-bla U2OS cells was used. This cell line stably expresses two fusion proteins: human GPR35 linked to a TEV protease site and a Gal4-VP16 transcription factor, and 13-arrestin/TEV protease fusion protein. The cell line also stably expresses the β-lactamase reporter gene under the control of a UAS response element. The activation of GPR35 by agonists leads to the recruitment of 13-arrestin/TEV protease fusion proteins to the activated GPR35. As a result, the protease cleaves the Gal4-VP16 transcription factor from the receptor, which then translocates to the nucleus and activates the expression of beta-lactamase. Briefly, 10000 cells per well were seeded in 384-well, black-wall, clear bottom assay plates with low fluorescence background (Corning), and cultured in DMEM (Invitrogen, 10569-010) supplemented with 10% dialyzed fetal bovine serum, 0.1 μM NEAA, 25 μM Hepes (pH 7.3), 100 U/ml Penicillin, and 100 μg/ml Streptomycin. After overnight culture, the cells were stimulated with ligands for 5 hrs in a humidified 37° C./5% CO₂, and then loaded with the cell permeable LiveBLAzer™ FRET B/G substrate. After the two hour incubation the coumarin:fluorescein ratio was measured using Tecan Safire II microplate reader (Männedorf, Switzerland). In the absence of beta-lactamase expression (i.e., no GPR35 activation), cells generate green fluorescence. In the presence of beta-lactamase expression upon receptor activation, the substrate is cleaved and the cells generate blue fluorescence. The coumarin:fluorescein ratio was used as a normalized reporter response.

### 2. Example 1

### Pharmacological Characterization of GPR35 Agonists

We combined DMR with Tango assay to pharmacologically characterize in detail the agonist activity and specificity of various ligands, according to a compound of the Formula (I), (II), or (III), against the GPR35. DMR assay is a whole cell phenotypic assay permitting detection of receptor-ligand interactions in native cells with wide pathway coverage and high sensitivity. For DMR assays, we used HT-29 cell line, a native cell line endogenously expresses functional GPR35. The Tango assay using U2OS-GPR35-bla cell line is an endpoint assay specific to the C-terminal modified GPR35 activation-induced O-arrestin translocation. For GPR35 DMR assay using HT-29 cell line seems to be sensitive to receptor activity associated with low receptor occupancy sate, while the Tango assay appears to be sensitive to receptor activity under high receptor occupancy state. Such a complementary approach is effective to discover novel GPR35 agonists.

DMR agonism profiling directly assesses the ability of nitrophenols to trigger DMR signals in native HT-29 cells.

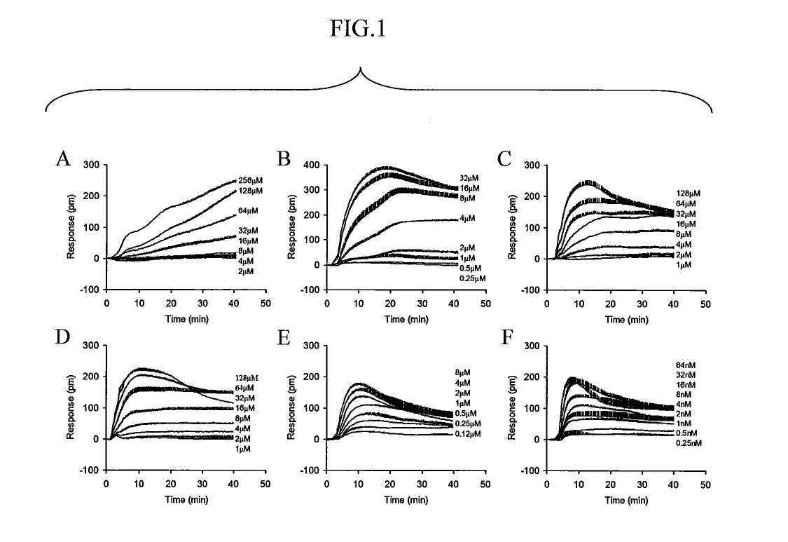

Referring to the Figures, FIGS. 1A to 1F demonstrate that a series of dinitrophenol analog compounds act as a GPR35 agonist.

Dinitrophenol is of the general formula:

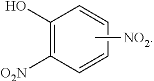

FIG. 1A shows the dynamic mass redistribution (DMR) of HT29 cells in response to stimulation at different doses with 2,5-dinitrophenol of the formula:

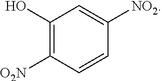 [SMILES: O=[N+]([O-])c1ccc([N+](=O)[O-])c(O)c1]

FIG. 1B shows the dynamic mass redistribution (DMR) of HT29 cells in response to stimulation at different doses with tolcapone (3,4-dihydroxy-4′-methyl-5-nitrobenzophenone) of the formula:

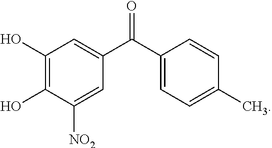 [SMILES: Cc1ccc(C(=O)c2cc(O)c(O)c([N+](=O)[O-])c2)cc1]

FIG. 1C shows the dynamic mass redistribution (DMR) of HT29 cells in response to stimulation at different doses with 2,4-dinitrophenol of the formula:

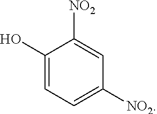 [SMILES: O=[N+]([O-])c1ccc(O)c([N+](=O)[O-])c1]

FIG. 1D shows the dynamic mass redistribution (DMR) of HT29 cells in response to stimulation at different doses with 3,5-dinitrocatechol of the formula:

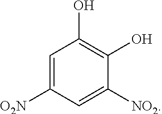 [SMILES: O=[N+]([O-])c1cc(O)c(O)c([N+](=O)[O-])c1]

FIG. 1E shows the dynamic mass redistribution (DMR) of HT29 cells in response to stimulation at different doses with nitecapone (3-[(3,4-dihydroxy-5-nitrophenyl)methylene]-2,4-pentanedione) of the formula:

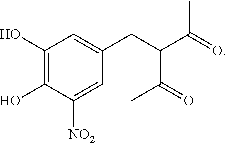 [SMILES: CC(=O)C(Cc1cc(O)c(O)c([N+](=O)[O-])c1)C(C)=O]

FIG. 1F shows the dynamic mass redistribution (DMR) of HT29 cells in response to stimulation at different doses with DC-Bi-DNP of the formula:

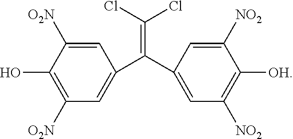 [SMILES: O=[N+]([O-])c1cc(C(=C(Cl)Cl)c2cc([N+](=O)[O-])c(O)c([N+](=O)[O-])c2)cc([N+](=O)[O-])c1O]

The data represents mean±standard deviation (s.d.) from two independent measurements, each in duplicate (n=4).

The results showed that all nitrophenols led to a clear dose-dependent and saturable DMR signal, but were diverse in their DMR characteristics, potency, and efficacy. FIGS. 1A to 1F show the real time dose responses of representative compounds. Among all nitrophenols tested, only 2,5-DNP led to a slowly increased DMR signal (FIG. 1A). Tolcapone resulted in a positive DMR with an early peak followed by a small decay to another elevated plateau (FIG. 1B); at the saturating doses its DMR amplitude was noticeably greater than those of zaprinast and all other nitrophenols (Table 1). All remaining compounds triggered a saturable DMR whose characteristics were almost identical to zaprinast, but whose amplitudes were ligand-dependent (FIGS. 1C to 1F, Table 1). The similarity in DMR characteristics suggests that these nitriphenols behaved as GPR35 agonists.

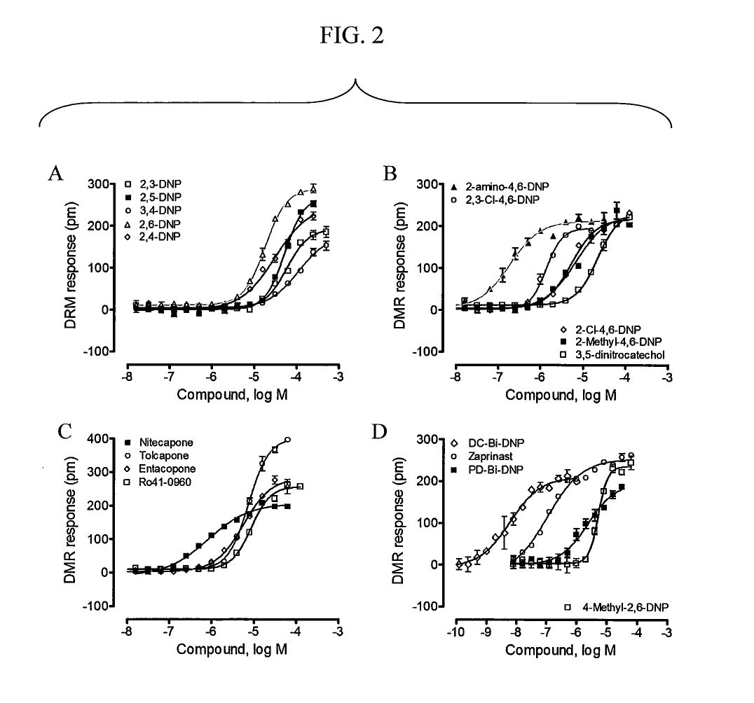

Next, we compared the relative potency of these compounds based on their DMR signals in HT-29 cells. FIGS. 2A to 2D show the dose-dependent DMR responses of a series of dinitrophenol analogs in HT29 cells. The DMR amplitude at 9 min post-stimulation was plotted as a function of the dose for each of the compounds. The data represents mean±s.d. from 2 independent measurements, each in duplicate (n=4).

Results showed that the dose responses of all compounds were best fitted with a monophasic sigmoidal non-linear regression. Except for 2,5-dinitrophenol (2,5-DNP) whose DMR amplitudes at 50 min poststimulation were used to calculate its potency, all other compounds were analyzed based on their maximal DMR amplitudes within 15 min poststimulation. The potency rank order, based on their EC50, was found to be DC-Bi-DNP (4,4′-(2,2-dichloroethene-1,1-diyl)bis(2,6-dinitrophenol)) (6 nM)>>zaprinast (0.11 μM)˜2-amino-4,6-DNP (0.19 μM)>>nitecapone (0.82 μM)>2,3-Cl-4,6-DNP (1.39 μM)>PD-Bi-DNP (4,4′-(propane-2,2-diyl)bis(2,6-dinitrophenol)) (2.35 μM)>4-methyl-2,6-DNP (5.27 μM)˜2-Cl-4,6-DNP (5.45 μM)˜entacapone (5.66 μM)>2-methyl-4,6-DNP (6.91 μM)˜tolcapone (7.44 μM)>Ro41-0960 (8.66 μM), 2,6-DNP, while the rest compounds gave rise to relatively low potency (>20 μM) (Table 1). SAR analysis showed that out of the five dinitrophenols 2,4-DNP and 2,6-DNP gave rise to comparatively high potency. Compared to 2,4-DNP, introduction of chloro, hydroxyl, methyl or amino group at its 6 position, or two cholo groups at its 5- and 6-positions all led to improved potency; and 2-amino-4,6-DNP exhibited a potency comparable to that of zaprinast. The four known CMOT inhibitors including the two clinically used drugs entacapone and tolcapone also displayed moderate potency at the GPR35; and among them nitecapone gave rise to the highest potency. For the three 2,6-DNP analogs, DC-Bi-DNP displayed nanomolar potency, the highest among all nitrophenols tested.

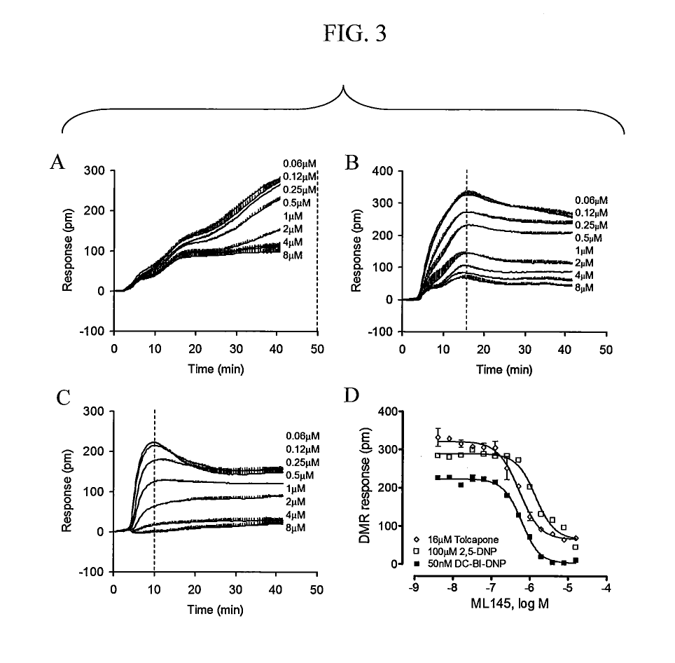

A mass of data suggests that a ligand-induced DMR is an integrative measure of cellular activity upon stimulation with the ligand. In parallel, many ligands may display polypharmacology, and can modulate multiple targets in the same cell line. Thus, we next determined the specificity of nitrophenol-induced DMR in HT-29 using a recently identified GPR35 potent antagonist ML-145 (Heynen-Genel S, et al. Antagonists for the orphan receptor GPR35. Probe Reports from the Molecular Libraries Program 2010, NBK5070). Here, cells were pretreated with ML-145 at different doses for 10 min, followed by stimulation with a nitrophenol compound at a fixed dose (Table 1). FIGS. 3A to 3D show that the known GPR35 antagonist ML145 dose-dependently inhibited the DMR signals induced by nitrophenol compounds in HT29.

FIG. 3A shows the partial inhibition of the DMR of 100 micromolar 2,4-dinitrophenol by ML145. FIG. 3B shows the partial inhibition of the DMR of 16 micromolar tolcapone by ML145. FIG. 3C shows the complete inhibition of the DMR of 50 nanomolar DC-Bi-DNP by ML145. FIG. 3D shows the dose-dependent inhibition of the DMR of three nitrophenol analogs by ML145, by GPR35 agonists caused internalization of GPR35 receptors in HT29 cells. The data represents mean±s.d. from 2 independent measurements, each in duplicate (n=2).

FIGS. 3A to 3D shows the effect of ML-145 on the DMR signals of three representative compounds. 2,5-DNP triggered a unique DMR among all nitrophenols, and ML-145 only partially inhibited the late response of 2,5-DNP in a dose-dependent manner, yielding an IC50 of 1.08±0.12 μM (2 independent measurements, n=4) (FIG. 3A). Tolcapone resulted in the greatest DMR in HT-29 among all nitrophenols tested, and ML-145 also only partially inhibited its DMR response with an IC50 of 0.54±0.04 μM (2 independent measurements, n=4) (FIG. 3B). Similarly, the DMR signal induced by 2,3-DNP, 2,4-DNP, 3,4-DNP, or Ro41-0960 was found to be partially inhibited by ML-145 in a dose-dependent manner, all of which resulted in comparable IC50 (Table 1). These results suggest that 2,3-DNP, 2,4-DNP, 3,4-DNP, 2,5-DNP, tolcapone and Ro41-0960 all activated another cellular receptor, besides the GPR35. Conversely, ML-145 fully inhibited the DMR of all other compounds including DC-Bi-DNP (FIG. 3C), suggesting that these compounds triggered DMR in HT-29 via the specific activation of endogenous GPR35.

Since β-arrestin plays an essential role in many GPCRs, we next examined the ability of nitrophenols to cause β-arrestin translocation-mediated gene reporter signal in U2OS-GPR35-bla cell line.

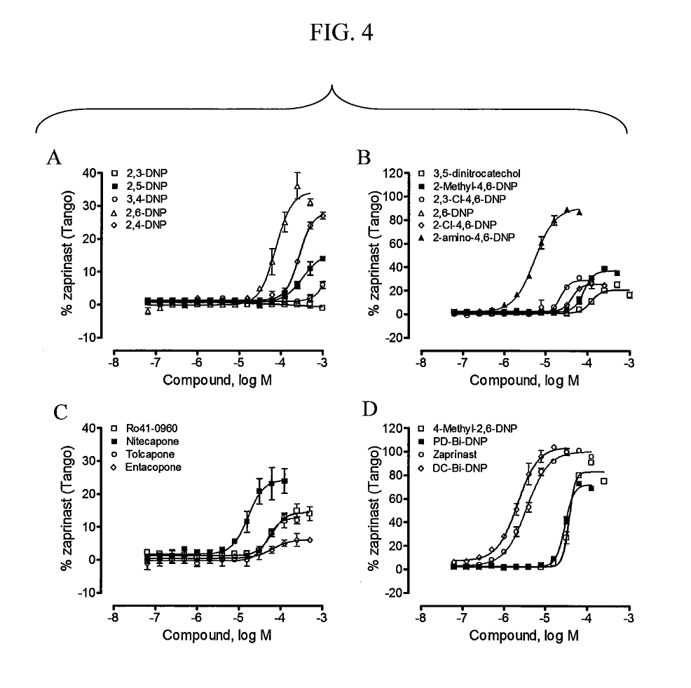

FIGS. 4A to 4D show the dose-dependent beta-arrestin translocation responses for a series of dinitrophenol analogs in U2OS-GPR35-bla cell line as measured using the Tango beta-arrestin assay. The Tango signals obtained were normalized with the maximal zaprinast response. The maximal zaprinast response was obtained within the same plate, and was set to be 100%. The data represents mean±s.d. from 2 independent measurements, each in duplicate (n=2). Zaprinast is of the formula:

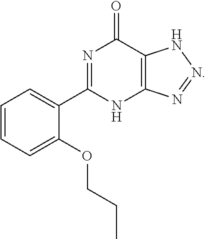 [SMILES: CCCOc1ccccc1-c1nc(=O)c2[nH]nnc2[nH]1]

Results showed that only 2,3-DNP up to 1 mM was inactive, and 3,4-DNP only showed little activity at 1 mM. All remaining nitrophenols resulted in a dose-dependent and saturable β-arrestin translocation signal. However, these nitrophenols were diverse in their potency and efficacy. Compared to DMR measurements, all nitrophenols displayed a right-shifted potency in Tango assays. The potency rank order obtained was: DC-Bi-DNP (2.12 μM)>zaprinast (3.25 μM)˜2-amino-4,6-DNP (5.37 μM)>>nitecapone (17.1 μM)>2,3-Cl-4,6-DNP (24.2 μM)>PD-Bi-DNP (29.2 μM)>4-methyl-2,6-DNP (36.4 μM)>2-Cl-4,6-DNP (41.7 μM)>tolcapone (49.2 μM)>Ro41-0960 (54.4 μM)>entacapone (65.6 μM)>2,6-DNP (76.6 μM)>2-methyl-4,6-DNP (81.1 μM)>3,5-dinitrocatechol (123 μM)>2,4-DNP (272 μM)˜2,5-DNP (331 μM). Such a potency rank order was mostly in agreement with that obtained using DMR assays, except for those ligands that are not specific to GPR35. Interestingly, the efficacies of these nitrophenols were also diverse. Using the maximal response of zaprinast within the same plate as a means to normalization, we found that the efficacy rank order was: zaprinast (100%)˜DC-Bi-DNP (104%)>4-methyl-2,6-DNP (83%)>2-amino-4,6-DNP (91%)>PD-Bi-DNP (72%)>2-methyl-4,6-DNP (37%)˜2,6-DNP (34%)˜2,3-Cl-4,6-DNP (32%)>2,4-DNP (28%)˜2-Cl-4,6-DNP (26%)˜tolcapone (25%)>3,5-dinitrocatechol (21%)>2,5-DNP (15%)˜Ro41-0960 (13%)˜nitecapone (13%)>entacapone (6%). Generally, the 2,6-DNP analogs, particularly its dimer compounds, gave rise to the higher efficacy, compared to 2,4-DNP and 3-nitrocatechol analogs.

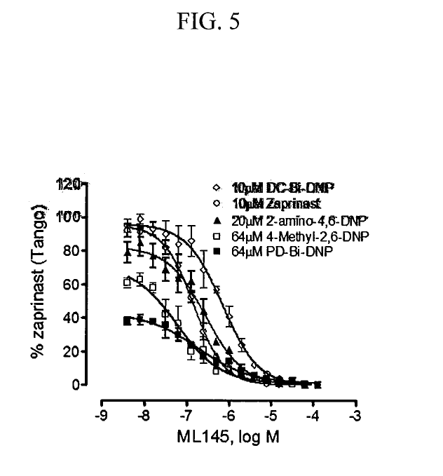

Lastly, we further determined the GPR35 specificity of the β-arrestin translocation signals of nitrophenols with relatively high efficacy. FIG. 5 shows that the known GPR35 antagonist ML145 dose-dependently inhibited the Tango beta-arrestin signals induced by selected nitrophenol compounds in U2OS-GPR35-bla cell line. The U2OS-GPR35-bla cells were pretreated with ML145 for 10 min, followed by stimulation with GPR35 agonists at a fixed concentration (indicated in the legend of FIG. 5). The data represents mean±s.d. from two independent measurements, each in duplicate (n=2). ML145 is of the formula:

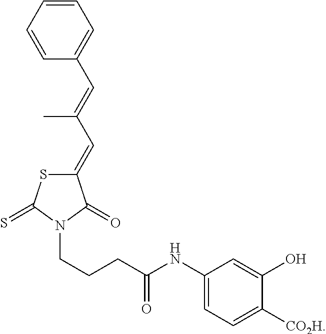 [SMILES: CC(/C=C1\SC(=S)N(CCCC(=O)Nc2ccc(C(=O)O)c(O)c2)C1=O)=C\c1ccccc1]

Results showed that ML-145 dose-dependently blocked the signals of four nitrophenols examined, similar to zaprinast. The 1050 was found to be 0.06±0.01 μM, 0.72±0.05 μM, 0.19±0.02 μM, 0.15±0.01 μM, 0.29±0.03 μM (2 independent measurements, n=4) for ML-145 to block the Tango signal induced by 64 μM 4-methyl-2,6-DNP, 10 μM DC-Bi-DNP, 64 μM DM-Bi-DNP, 10 μM Zaprinast, and 20 μM 2-amino-4,6-DNP, respectively. Together with Tango agonist assay, these results suggest that these nitrophenols activates GPR35 in the engineered cell line and cause β-arrestin translocation.

Using the same approach, we found that Bi-ASA ((E)-(5,5-diazene-1,2-diyl)bis(2-hydroxybenzoic acid)), olsalazine, sulfasalazine, 5-aminosalicylic acid (5-ASA), balsalazine are also GPR35 agonists. All of aminosalicyclic acid analogs triggered a dose-dependent DMR in HT-29 cells, yielding an EC50 of 2.09 nM, 14.7 μM, 2.99 μM, 2.59 μM, and 21.8 nM for Bi-ASA, 5-ASA, sulfasalazine, balsalazide, and olsalazine, respectively. Furthermore, all ASA analogs also led to a dose-dependent beta-arrestin translocation signal in U2OS-GPR35-bla cell lines, yielding an EC50 of 1.70 μM, 545 μM, 31.3 μM, 46.2 μM, and 1.26 μM for Bi-ASA, 5-ASA, sulfasalazine, balsalazide, and olsalazine, respectively. Compared to zaprinast, Bi-ASA acted as a strong partial agonist, 5-ASA was a weak partial agonist, sulfasalazine was a partial agonist, balsalazide and olasalazine were full agonists, based on the maximal responses obtained using Tango assays. In addition, both DMR and Tango antagonist assays showed that ML145 can dose dependently block the DMR signal and Tango signal of either ASA analog, each at its respective EC80. In summary, these ligands are GPR35 agonists. Interestingly, 5-ASA, balsalazide, and olsalazine are clinically used to treat inflammatory bowel disease, such as ulcerative colitis and mild-to-moderate Crohn's disease. However, their molecular mode of action is unknown. The potent GPR35 agonist activity of these drugs at GPR35 suggest that GPR35 is a druggable target for treating treat inflammatory bowel disease, such as ulcerative colitis and mild-to-moderate Crohn's disease.

Compounds and their pharmacological characteristics are listed in Table 1. The EC₅₀ was obtained using DMR agonism and Tango assays, while the IC_{50, ML145} was for ML-145 to block the DMR of each agonist at a fixed dose, and their efficacy was the maximal responses of each ligand measured using DMR and the maximal response after normalized to the zaprinast maximal response obtained using Tango assays.

TABLE 1

| | | | | Efficacy** | |
|---|---|---|---|---|---|
| | Potency (μM)* | | | DMR | Tango |
| Compound | EC_{50, DMR} | EC_{50, Tango} | IC_{50, MT145}**** | (pm) | (% zaprinast) |
| 2,3-DNP | 58.5 ± 4.7 | inactive | 0.37 ± 0.02*** | 192 ± 13 | inactive |
| 2,4-DNP | 31.1 ± 2.9 | 272 ± 27 | 0.31 ± 0.02*** | 249 ± 17 | 28 ± 1 |
| 2,5-DNP | 52.6 ± 4.7** | 331 ± 35 | 1.08 ± 0.12*** | 269 ± 15 | 15 ± 1 |
| 2,6-DNP | 18.2 ± 1.0 | 76.0 ± 6.1 | 0.92 ± 0.08 | 288 ± 12 | 34 ± 4 |
| 3,4-DNP | 112 ± 15 | >500 | 0.43 ± 0.03*** | 178 ± 12 | >6 |
| 3,5-dinitrocatechol | 22.0 ± 1.9 | 123 ± 12 | 0.84 ± 0.10 | 232 ± 14 | 21 ± 1 |
| 2-Cl-4,6-DNP | 5.45 ± 0.37 | 41.7 ± 3.9 | 0.077 ± 0.008 | 217 ± 15 | 26 ± 2 |
| 2,3-Cl-4,6-DNP | 1.39 ± 0.11 | 24.2 ± 2.1 | 0.11 ± 0.01 | 195 ± 11 | 32 ± 4 |
| 2-Methyl-4,6-DNP | 6.91 ± 0.57 | 81.1 ± 7.9 | 0.23 ± 0.02 | 229 ± 14 | 37 ± 2 |
| 2-Amino-4,6-DNP | 0.19 ± 0.01 | 5.37 ± 0.41 | 0.18 ± 0.01 | 212 ± 17 | 91 ± 6 |
| Ro41-0960 | 8.66 ± 0.71 | 54.4 ± 4.3 | 0.92 ± 0.07*** | 257 ± 16 | 13 ± 1 |
| Entacapone | 5.66 ± 0.44 | 65.6 ± 5.6 | 0.14 ± 0.01 | 283 ± 19 | 6 ± 1 |
| Tolcapone | 7.44 ± 0.59 | 49.2 ± 4.2 | 0.54 ± 0.04*** | 400 ± 33 | 25 ± 2 |
| Nitecapone | 0.82 ± 0.06 | 17.1 ± 1.3 | 0.21 ± 0.2 | 207 ± 14 | 13 ± 1 |
| 4-Methyl-2,6-DNP | 5.27 ± 0.47 | 36.4 ± 2.8 | 0.85 ± 0.07 | 235 ± 13 | 83 ± 5 |
| DC-Bi-DNP | 0.006 ± 0.001 | 2.12 ± 0.14 | 0.60 ± 0.05 | 210 ± 11 | 104 ± 6 |
| PD-Bi-DNP | 2.35 ± 0.17 | 29.2 ± 2.3 | 0.30 ± 0.03 | 195 ± 12 | 72 ± 5 |
| Zaprinast | 0.11 ± 0.01 | 3.25 ± 0.28 | 0.43 ± 0.03 | 254 ± 14 | 100 ± 9 |

*All data represents mean ± s.d. from 2 independent measurements (n = 4).
**The DMR amplitudes at 50 min post stimulation were analyzed for 2,5-DNP, while the maximal DMR amplitudes were used for all other compounds.
***ML-145 partially inhibited the DMR of these compounds in a dose-dependent manner.
****The IC_{50, ML145} was obtained for each agonist at a fixed dose: 200 μM 2,3-DNP, 128 μM 2,4-DNP, 100 μM 2,5-DNP, 20 μM 2,6-DNP, 50 μM 3,4-DNP, 32 μM 3,5-dinitrocatechol, 250 nM 2-amino-4,6-DNP, 32 μM Ro41-0960, 32 μM 4-methyl-2,6-DNP, 50 nM DC—Bi—DNP, 16 μM PD—Bi—DNP, 1 μM zaprinast, and 16 μM for all other compounds.

Using the same approach, we found that Bi-ASA ((E)-(5,5-diazene-1,2-diyl)bis(2-hydroxybenzoic acid)), olsalazine, sulfasalazine, 5-aminosalicylic acid (5-ASA), balsalazine are also GPR35 agonists. All of the amino alicyclic acid analogs triggered a dose-dependent DMR in HT-29 cells, yielding an EC50 of 2.09 nM, 14.7 μM, 2.99 μM, 2.59 μM, and 21.8 nM for Bi-ASA, 5-ASA, sulfasalazine, balsalazide, and olsalazine, respectively. Furthermore, all ASA analogs also led to a dose-dependent beta-arrestin translocation signal in U2OS-GPR35-bla cell lines, yielding an EC50 of 1.70 μM, 545 μM, 31.3 μM, 46.2 μM, and 1.26 μM for Bi-ASA, 5-ASA, sulfasalazine, balsalazide, and olsalazine, respectively. Compared to zaprinast, Bi-ASA acted as a strong partial agonist, 5-ASA was a weak partial agonist, sulfasalazine was a partial agonist, balsalazide and olasalazine were full agonists, based on the maximal responses obtained using Tango assays. In addition, both DMR and Tango antagonist assays showed that ML145 can dose dependently block the DMR signal and Tango signal of either ASA analog, each at its respective EC80. In summary, these ligands are GPR35 agonists. Interestingly, 5-ASA, balsalazide, and olsalazine are clinically used to treat inflammatory bowel disease, such as ulcerative colitis and mild-to-moderate Crohn's disease. However, their molecular mode of action is unknown. The potent GPR35 agonist activity of these drugs at GPR35 suggest that GPR35 is a druggable target for treating treat inflammatory bowel disease, such as ulcerative colitis and mild-to-moderate Crohn's disease.

Similarly, we found that pyrogallol, 3-methoxycatechol, propyl gallate, and octyl gallate were active to trigger DMR signal in HT-29 cells that can be blocked by ML-145, and also trigger Tango signal in U2OS-GPR35-bla cells that can be blocked by ML-15, suggesting that all these compounds are also GPR35 agonists.

### E. SEQUENCES

The protein sequence of GPR35a (UniProtKB/Swiss-Prot:Q9HC97) (SEQ ID NO:1) is

MNGTYNTCGSSDLTWPPAIKLGFYAYLGVLLVLGLLLNSLALWVFCCRMQ
QWTETRIYMTNLAVADLCLLCTLPFVLHSLRDTSDTPLCQLSQGIYLTNR
YMSISLVTAIAVDRYVAVRHPLRARGLRSPRQAAAVCAVLWVLVIGSLVA
RWLLGIQEGGFCFRSTRHNFNSMAFPLLGFYLPLAVVVFCSLKVVTALAQ
RPPTDVGQAEATRKAARMVWANLLVFVVCFLPLHVGLTVRLAVGWNACAL
LETIRRALYITSKLSDANCCLDAICYYYMAKEFQEASALAVAPSAKAHKS
QDSLCVTLA

The Homo sapiens G protein-coupled receptor 35a (GPR35a), mRNA (NCBI Reference Sequence: NM_{—}005301.2) (SEQ ID NO:2) is

| | |
|---|---|
| 1 | caggccagag tcccagctgt cctggactct gctgtgggga |
| | agggctgatg caggtgtgga |
| 61 | gtcaaatgtg ggtgcctcct gcagccgggt gccaggaggg |
| | gtggaggggc caccctgggc |
| 121 | tttgtccggg agcctggtct tcccgtcctt gggctgacag |
| | gtgctgctgc ctctgagccc |
| 181 | tccctgctaa gagctgtgtg ctgggtaagg ctggtggccc |
| | tttgggctcc ctgtccagga |
| 241 | tttgtgctct ggagggtagg gcttgctggg ctggggactg |
| | gaggggaacg tggagctcct |
| 301 | tctgcctcct ttcctgcccc atgacagcag gcagatccca |
| | ggagagaaga gctcaggaga |
| 361 | tgggaagagg atctgtccag gggttagacc tcaagggtga |
| | cttggagttc tttacggcac |
| 421 | ccatgctttc tttgaggagt tttgtgtttg tgggtgtggg |
| | gtcggggctc acctcctccc |
| 481 | acatccctgc ccagaggtgg gcagagtggg ggcagtgcct |
| | tgctccccct gctcgctctc |
| 541 | tgctgacctc cggctccctg tgctgcccca ggaccatgaa |
| | tggcacctac aacacctgtg |
| 601 | gctccagcga cctcacctgg cccccagcga tcaagctggg |
| | cttctacgcc tacttgggcg |
| 661 | tcctgctggt gctaggcctg ctgctcaaca gcctggcgct |
| | ctgggtgttc tgctgccgca |
| 721 | tgcagcagtg gacggagacc cgcatctaca tgaccaacct |
| | ggcggtggcc gacctctgcc |
| 781 | tgctgtgcac cttgcccttc gtgctgcact ccctgcgaga |
| | cacctcagac acgccgctgt |
| 841 | gccagctctc ccagggcatc tacctgacca acaggtacat |
| | gagcatcagc ctggtcacgg |
| 901 | ccatcgccgt ggaccgctat gtggccgtgc ggcacccgct |
| | gcgtgcccgc gggctgcggt |
| 961 | cccccaggca ggctgcggcc gtgtgcgcgg tcctctgggt |
| | gctggtcatc ggctccctgg |
| 1021 | tggctcgctg gctcctgggg attcaggagg gcggcttctg |
| | cttcaggagc acccggcaca |
| 1081 | atttcaactc catggcgttc ccgctgctgg gattctacct |
| | gcccctggcc gtggtggtct |
| 1141 | tctgctccct gaaggtggtg actgccctgg cccagaggcc |
| | acccaccgac gtggggcagg |
| 1201 | cagaggccac ccgcaaggct gcccgcatgg tctgggccaa |
| | cctcctggtg ttcgtggtct |
| 1261 | gcttcctgcc cctgcacgtg gggctgacag tgcgcctcgc |
| | agtgggctgg aacgcctgtg |
| 1321 | ccctcctgga gacgatccgt cgcgccctgt acataaccag |
| | caagctctca gatgccaact |
| 1381 | gctgcctgga cgccatctgc tactactaca tggccaagga |
| | gttccaggag gcgtctgcac |
| 1441 | tggccgtggc tcccagtgct aaggcccaca aaagccagga |
| | ctctctgtgc gtgaccctcg |
| 1501 | cctaagaggc gtgctgtggg cgctgtgggc caggtctcgg |
| | gggctccggg aggtgctgcc |
| 1561 | tgccagggga agctggaacc agtagcaagg agcccgggat |
| | cagccctgaa ctcactgtgt |
| 1621 | attctcttgg agccttgggt gggcagggac ggcccaggta |
| | cctgctctct tgggaagaga |
| 1681 | gagggacagg gacaagggca agaggactga ggccagagca |
| | aggccaatgt cagagacccc |
| 1741 | cgggatgggg cctcacactt gccaccccca gaaccagctc |
| | acctggccag agtgggttcc |
| 1801 | tgctggccag ggtgcagcct tgatgacacc tgccgctgcc |
| | cctcggggct ggaataaaac |
| 1861 | tccccaccca gagtc |

The cDNA sequence for GPR35a (SEQ ID NO:3):

```
...................................ATGAATGGCACCTACAACACCTGTG
        26
GCTCCAGCGACCTCACCTGGCCCCCAGCGATCAAGCTGGGCTTCTACGCCTACTTGGGCG
        86
TCCTGCTGGTGCTAGGCCTGCTGCTCAACAGCCTGGCGCTCTGGGTGTTCTGCTGCCGCA
      146
TGCAGCAGTGGACGGAGACCCGCATCTACATGACCAACCTGGCGGTGGCCGACCTCTGCC
      206
TGCTGTGCACCTTGCCCTTCGTGCTGCACTCCCTGCGAGACACCTCAGACACGCCGCTGT
      266
GCCAGCTCTCCCAGGGCATCTACCTGACCAACAGGTACATGAGCATCAGCCTGGTCACGG
      326
CCATCGCCGTGGACCGCTATGTGGCCGTGCGGCACCCGCTGCGTGCCCGCGGGCTGCGGT
      386
CCCCCAGGCAGGCTGCGGCCGTGTGCGCGGTCCTCTGGGTGCTGGTCATCGGCTCCCTGG
      446
TGGCTCGCTGGCTCCTGGGGATTCAGGAGGGCGGCTTCTGCTTCAGGAGCACCCGGCACA
      506
ATTTCAACTCCATGGCGTTCCCGCTGCTGGGATTCTACCTGCCCCTGGCCGTGGTGGTCT
      566
TCTGCTCCCTGAAGGTGGTGACTGCCCTGGCCCAGAGGCCACCCACCGACGTGGGGCAGG
      626
CAGAGGCCACCCGCAAGGCTGCCCGCATGGTCTGGGCCAACCTCCTGGTGTTCGTGGTCT
      686
GCTTCCTGCCCCTGCACGTGGGGCTGACAGTGCGCCTCGCAGTGGGCTGGAACGCCTGTG
      746
CCCTCCTGGAGACGATCCGTCGCGCCCTGTACATAACCAGCAAGCTCTCAGATGCCAACT
      806
GCTGCCTGGACGCCATCTGCTACTACTACATGGCCAAGGAGTTCCAGGAGGCGTCTGCAC
      866
TGGCCGTGGCTCCCAGTGCTAAGGCCCACAAAAGCCAGGACTCTCTGTGCGTGACCCTCG
      926
CCTAA.................................................................
```

The protein sequence of GPR35b (SEQ ID NO:4) (S. Okumura, H. Baba, T. Kumada, K. Nanmoku, H. Nakajima, Y. Nakane, K. Hioki, K. Ikenaka (2004) Cloning of a G-protein-coupled receptor that shows an activity to transform NIH3T3 cells and is expressed in gastric cancer cells, Cancer Sci. 95: 131-135) is

MLSGSRAVPTPHRGSEELLKYMLHSPCVSLTMNGTYNTCGSSDLTWPPAI
KLGFYAYLGVLLVLGLLLNSLALWVFCCRMQQWTETRIYMTNLAVADLCL
LCTLPFVLHSLRDTSDTPLCQLSQGIYLTNRYMSISLVTAIAVDRYVAVR
HPLRARGLRSPRQAAAVCAVLWVLVIGSLVARWLLGIQEGGFCFRSTRHN
FNSMAFPLLGFYLPLAVVVFCSLKVVTALAQRPPTDVGQAEATRKAARMV
WANLLVFVVCFLPLHVGLTVRLAVGWNACALLETIRRALYITSKLSDANC
CLDAICYYYMAKEFQEASALAVAPRAKAHKSQDSLCVTLA

The cDNA sequence of GPR35b (SEQ ID NO:5) is

```
          1
ATGCTGAGTGGTTCCCGGGCTGTCCCCACTCCACACCGTGGCAGTGAAGAGCTGCTGAAG
        61
TACATGCTTCATAGTCCTTGCGTCTCTCTGACCATGAATGGCACCTACAACACCTGTGGC
      121
TCCAGCGACCTCACCTGGCCCCCAGCGATCAAGCTGGGCTTCTACGCCTACTTGGGCGTC
      181
CTGCTGGTGCTAGGCCTGCTGCTCAACAGCCTGGCGCTCTGGGTGTTCTGCTGCCGCATG
      241
CAGCAGTGGACGGAGACCCGCATCTACATGACCAACCTGGCGGTGGCCGACCTCTGCCTG
      301
CTGTGCACCTTGCCCTTCGTGCTGCACTCCCTGCGAGACACCTCAGACACGCCGCTGTGC
      361
CAGCTCTCCCAGGGCATCTACCTGACCAACAGGTACATGAGCATCAGCCTGGTCACGGCC
      421
ATCGCCGTGGACCGCTATGTGGCCGTGCGGCACCCGCTGCGTGCCCGCGGGCTGCGGTCC
      481
CCCAGGCAGGCTGCGGCCGTGTGCGCGGTCCTCTGGGTGCTGGTCATCGGCTCCCTGGTG
      541
GCTCGCTGGCTCCTGGGGATTCAGGAGGGCGGCTTCTGCTTCAGGAGCACCCGGCACAAT
      601
TTCAACTCCATGGCGTTCCCGCTGCTGGGATTCTACCTGCCCCTGGCCGTGGTGGTCTTC
      661
TGCTCCCTGAAGGTGGTGACTGCCCTGGCCCAGAGGCCACCCACCGACGTGGGGCAGGCA
      721
GAGGCCACCCGCAAGGCTGCCCGCATGGTCTGGGCCAACCTCCTGGTGTTCGTGGTCTGC
      781
TTCCTGCCCCTGCACGTGGGGCTGACAGTGCGCCTCGCAGTGGGCTGGAACGCCTGTGCC
      841
CTCCTGGAGACGATCCGTCGCGCCCTGTACATAACCAGCAAGCTCTCAGATGCCAACTGC
      901
TGCCTGGACGCCATCTGCTACTACTACATGGCCAAGGAGTTCCAGGAGGCGTCTGCACTG
      961
GCCGTGGCTCCCAGTGCTAAGGCCCACAAAAGCCAGGACTCTCTGTGCGTGACCCTCGCC
    1021
TAA
```

### F. REFERENCES

1. U.S. Application No. 61/365,861. Fang, Y., Deng, H., Hu, H., Sun, H., He, M., Niu, W. Compositions and methods for the treatment of pathological conditions related to GPR35 and/or GPR35-hERG complex.
2. US20070077602 A1 “GPR35 and modulators thereof for the treatment of metabolic-related disorders”
3. WO2011011235 A1 “TREATMENT OF DISORDERS ASSOCIATED WITH G PROTEIN-COUPLED RECEPTOR 35 (GPR35)”
4. WO2005085867 A8 “Screening method for emulators of neural activity and digestive system using gpr35”
5. WO2005059546 A3 “DIAGNOSTICS AND THERAPEUTICS FOR DISEASES ASSOCIATED WITH G PROTEIN-COUPLED RECEPTOR 35 (GPR35)”
6. J. Guo, D. J. Williams, H. L. Puhl III, S. R. Ikeda (2008) Inhibition of N-type calcium channels by activation of GPR35, an orphan receptor, heterologously expressed in rat sympathetic neurons, J. Pharmacol. Exp. Ther. 324: 342-351
7. Jenkins, L., Brea, J., Smith, N. J., Hudson, B. D., Reilly, G., Bryant, N. J., Castro, M., Loza, M.-I., Milligan, G. (2010) Identification of novel species-selective agonists of the G-protein-coupled receptor GPR35 that promote recruitment of β-arrestin-2 and activate Gα13. Biochemical Journal 432, 451-459
8. B. F. O'Dowd, T. Nguyen, A. Marchese, R. Cheng, K. R. Lynch, H. H. Heng, L. F. Kolakowski Jr, S. R. George (1998) Discovery of three novel G-protein-coupled receptor genes, Genomics 47: 310-313
9. S. Oka, R. Ota, M. Shima, A. Yamashita, T. Sugiura (2010) GPR35 is a novel lysophosphatidic acid receptor. Biochem. Biophys. Res. Comm. 395: 232-237
10. S. Okumura, H. Baba, T. Kumada, K. Nanmoku, H. Nakajima, Y. Nakane, K. Hioki, K. Ikenaka (2004) Cloning of a G-protein-coupled receptor that shows an activity to transform NIH3T3 cells and is expressed in gastric cancer cells, Cancer Sci. 95: 131-135
11. H. Ohshiro, H. Tonai-Kachi, K. Ichikawa (2008) GPR35 is a functional receptor in rat dorsal root ganglion neurons, Biochem. Biophys. Res. Commun. 365: 344-348.
12. K. D. Min, M. Asakura, Y. Liao, K. Nakamaru, H. Okazaki, T. Takahashi, K. Fujimoto, S. Ito, A. Takahashi, H. Asanuma, S. Yamazaki, T. Minamino, S. Sanada, O. Seguchi, A. Nakano, Y. Ando, T. Otsuka, H. Furukawa, T. Isomura, S. Takashima, N. Mochizuki, M. Kitakaze (2010) Identification of genes related to heart failure using global gene expression profiling of human failing myocardium, Biochem. Biophys. Res. Commun. 393: 55-60.
13. A. E. Shrimpton, B. R. Braddock, L. L. Thomson, C. K. Stein, J. J. Hoo (2004) Molecular delineation of deletions on 2q37.3 in three cases with an Albright hereditary osteodystrophy-like phenotype, Clin. Genet. 66: 537-544
14. J. Wang, N. Simonavicius, X. Wu, G. Swaminath, J. Reagan, H. Tian, L. Ling, (2006) Kynurenic acid as a ligand for orphan G protein-coupled receptor GPR35, J. Biol. Chem. 281: 22021-22028;

What is claimed is:

1. A method for treatment or prevention of a disease, comprising
administering, to a subject diagnosed as in need of such treatment or prevention, an effective amount of a compound of the Formula (I), (II), or (III): 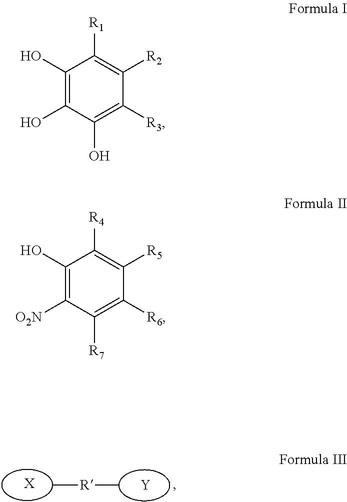
where:
R¹, R², R³, R⁴, R⁵, R⁶, and R⁷ are each independently selected from —H, —OH, —NO₂, —C(═O)OH, —C(═O)OR″ where R″ is substituted or unsubstituted alkyl having from 1 to 20 carbon atoms, halide, acyl halide, substituted or unsubstituted alkyl, substituted or unsubstituted —NH-alkyl, substituted or unsubstituted —CH₂—NH—C(═O)—R″, substituted or unsubstituted —CH═NH—NH—C(═O)—NH—R″, substituted or unsubstituted —CH═NH—NH—C(═S)—NH—R″, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, or a substituted or unsubstituted alkoxy;
optionally R² and R³ taken together form a substituted or unsubstituted aryl, substituted or unsubstituted heteroaryl, substituted or unsubstituted cycloalkenyl, or substituted or unsubstituted heterocyclyl;
optionally R⁴ and R⁵, R⁵ and R⁶, or R⁶ and R⁷, taken together form a substituted or unsubstituted aryl, substituted or unsubstituted heteroaryl, substituted or unsubstituted cycloalkenyl or substituted or unsubstituted heterocyclyl;
in Formula (III), R′ is a divalent moiety selected from: a covalent carbon-carbon bond, —N═N-(cis- or trans-), —NH—NH—, —O—, —(CH₂CH₂O)ₙ— or —(CH₂CH(—CH₃)—O)ₙ— where n is from 1 to 10, substituted or unsubstituted alkyl, substituted or unsubstituted —NH-alkyl, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, substituted or unsubstituted alkoxy, or of the formulas —CH═CH—C(═O)—CH═C(—OH)—CH═CH— (conjugated keto-enol form) or —CH═CH—C(═O)—O—CH₂—CH₂— (conjugated ester);
in Formula (III), X and Y are independently selected from substituents of the Formulas (IV), (V), (VI), (VII), (VIII), or (IX): 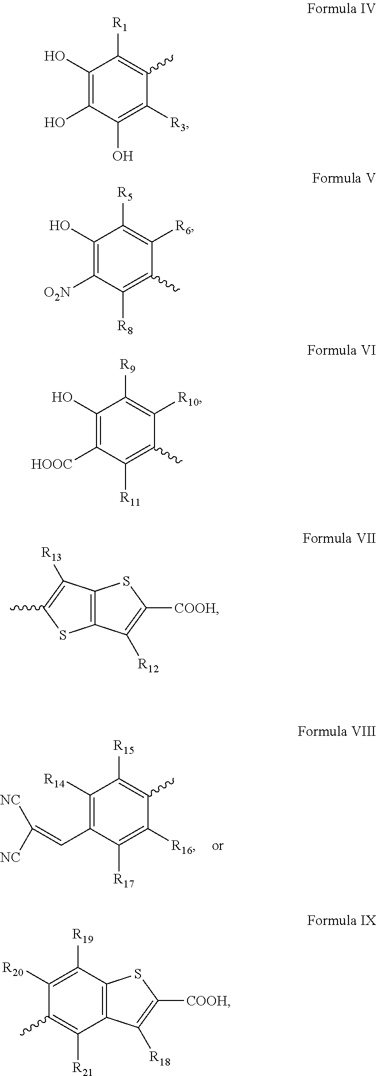
where
the wavy or squiggly line represents a connecting valence to R′ or to another of X or Y when R′ is a single covalent carbon-carbon bond, and in any of the Formulas (IV), (V), (VI), (VII), (VIII), or (IX), each R¹, R³, R⁵, R⁶, R⁸, R⁹, R¹⁰, R¹¹, R¹², R¹³, R¹⁴, R¹⁵, R¹⁶, R¹⁷, R¹⁸, R¹⁹, R²⁰, and R²¹ is independently selected from —H, —OH, halide, acyl halide, substituted or unsubstituted alkyl, substituted or unsubstituted —NH-alkyl, substituted or unsubstituted alkynyl, substituted or unsubstituted alkenyl, substituted or unsubstituted aryl, substituted or unsubstituted —O-aryl, substituted or unsubstituted heteroaryl, substituted or unsubstituted alkylaryl, substituted or unsubstituted cycloalkyl, substituted or unsubstituted heterocyclyl, or a substituted or unsubstituted alkoxy; or a pharmaceutically acceptable salt, solvate, clathrate, or prodrug thereof.

2. The method of claim 1, wherein R′ is a covalent carbon-carbon single bond, —CH₂—, —CH═CH—, —CH₂CH(CN)—, —CH₂CH(OH)—, —CH₂C(═O)—, —N═N—, —NH—NH—, —C(CH₃)₂—, —C(═CCl₂)—, —CH₂CH(NH₂)—, —CH₂C(CH₃)(NH₂)—, —(CH₂CH₂O)ₙ— or —(CH₂CH(CH₃)O)ₙ— where n is from 1 to 10.

3. The method of claim 2, where R′ is —(CH₂CH₂O)ₙ— n is 1 to 7.

4. The method of claim 1, wherein the compound of Formula (I) has R² and R³ taken together is a heterocyclyl.

5. The method of claim 1, wherein the compound of Formula (II) has R⁵ and R⁶ taken together is a heterocyclyl.

6. The method of claim 1, wherein the compound of the Formula (I) is selected from: 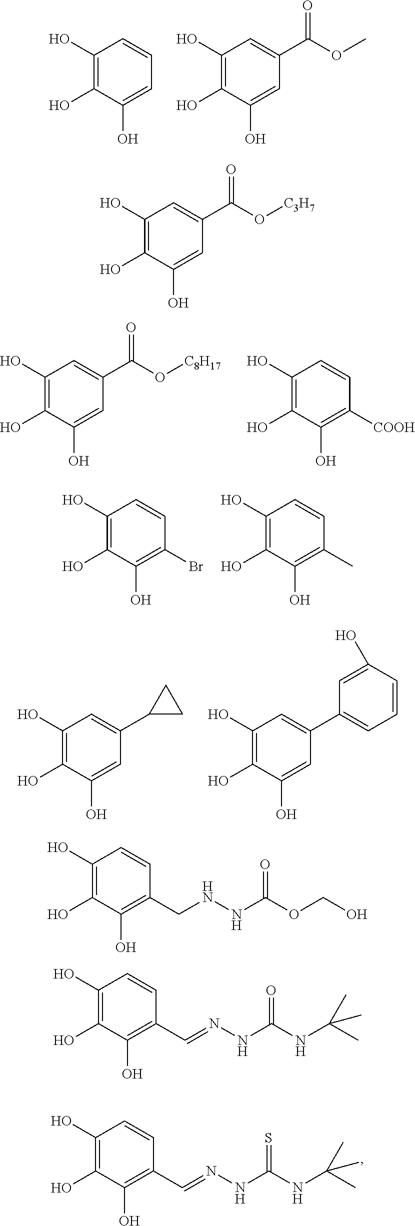
or a mixture thereof.

7. The method of claim 1, wherein the compound of the Formula (II) is selected from: 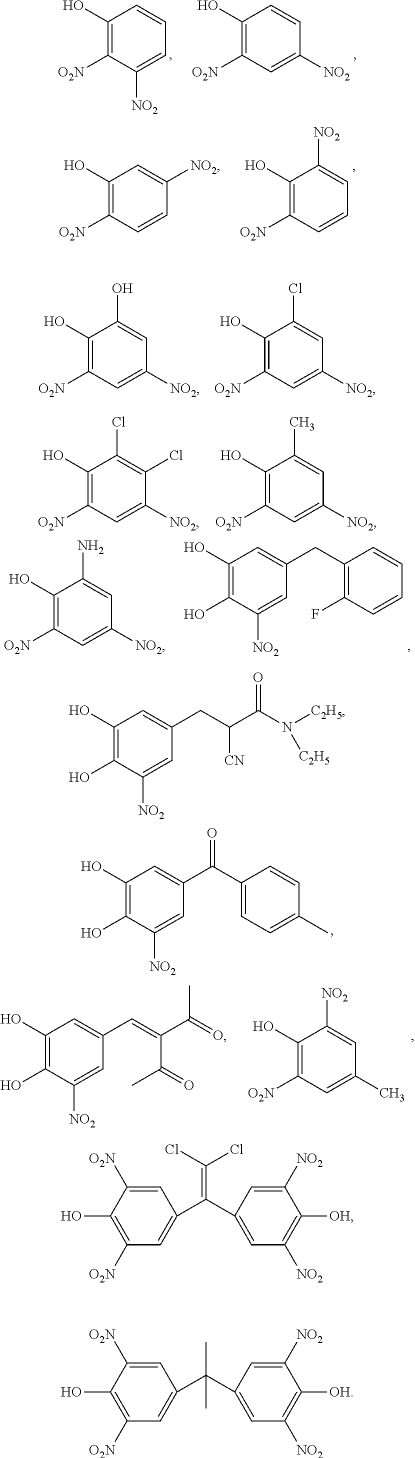
or a mixture thereof.

8. The method of claim 1, wherein the compound of the Formula (III) is selected from: 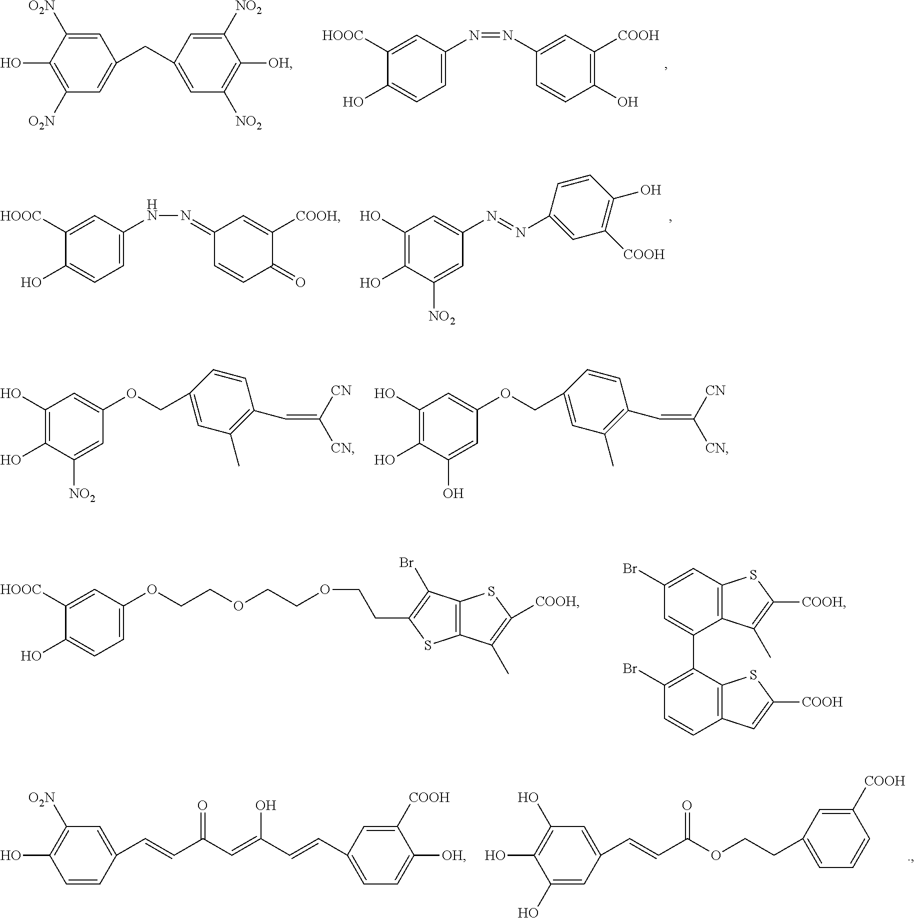
or a mixture thereof.

9. The method of claim 1, wherein the compound of the Formula (I), (II), or (III), is a GPR35 modulator.

10. The method of claim 1, wherein the disease is inflammation, metabolic disorder, inflammatory bowel disorder, congestive heart failure, or cancer.

11. The method of claim 10, wherein the metabolic disorder is diabetes, Type I diabetes, Type II diabetes, inadequate glucose tolerance, insulin resistance, hyperglycemia, hyperinsulinemia, hyperlipidemia, hypertriglyceridemia, hypercholesterolemia, dyslipidemia, obesity, premature or accelerated aging, Syndrome X, atherosclerosis, heart disease, stroke, hypertension, and peripheral vascular disease.

12. The method of claim 10, wherein the cancer is selected from prostate, leukemia, hormone dependent type, breast, colon, lung, epidermal, liver, esophageal, stomach, brain, and kidney.

13. The method of claim 10, wherein the inflammatory bowel disorder is selected from ulcerative colitis and Crohn's disease.

14. The method of claim 1, wherein the administering is accomplished by at least one of: rectal, buccal, sublingual, intravenous, subcutaneous, intradermal, transdermal, intraperitoneal, oral, eye drops, parenteral, and topically, or a combination thereof.

15. A pharmaceutical composition including a compound of claim 1 of the Formula (I), (II), or (III), or a pharmaceutically acceptable salt, solvate, clathrate, or prodrug thereof.
